(19) United States
(12) Patent Application Publication
(10) Pub. No.: US 2013/0274712 A1
(43) Pub. Date: Oct. 17, 2013

(54) HAPTIC SYSTEM FOR BALLOON TIPPED CATHETER INTERVENTIONS

(71) Applicant: Stuart O. Schecter, Great Neck, NJ (US)

(72) Inventor: Stuart O. Schecter, Great Neck, NJ (US)

(21) Appl. No.: 13/667,644

(22) Filed: Nov. 2, 2012

Related U.S. Application Data
(60) Provisional application No. 61/628,551, filed on Nov. 2, 2011.

(51) Int. Cl. A61M 25/10

(52) U.S. Cl. CPC A61M 25/10
USPC 604/510; 604/96.01; 604/101.01; 604/100.01

(57) ABSTRACT

Embodiments of this invention include hand-held handles and systems for balloon-tipped catheter interventions that enable a user to appreciate a combination of palpable sensations as though his or her hand is actual anatomic tissue in situ in real time. The system may include a haptic handle coupled to the proximal end of the catheter where the haptic handle includes a balloon-shaped haptic interface that exhibits geometric characteristics reflecting an anatomy of the interventional balloon and provides tangible sensations representative of tissues surrounding the interventional balloon. Multiple sensors and actuators may be used to create a non-virtual, transparent experience communicated to a user with a three dimensional, volumetric haptic display. In one embodiment, the sensors are positioned about or within an inflatable balloon positioned at the distal aspect of an inserted catheter used to treat cardiac and vascular diseases such as coronary or peripheral vascular occlusion and ablation of cardiac tissue.

### RELATED APPLICATION

The present application claims the benefit of U.S. Provisional Application No. 61/628,551 filed Nov. 2, 2011, which is incorporated herein in its entirety by reference.

### FIELD OF THE INVENTION

The present invention generally relates to a minimally-invasive, hand-held surgical interface that provides real time haptic feedback of tissue mechanics and intra-cardiac fluid dynamics. More particularly, embodiments of the present invention relate to a surgical haptic handle and system useful to provide the operator haptic feedback in therapy interventions involving a balloon tip catheter.

### BACKGROUND OF THE INVENTION

As conventionally designed cardiac catheters course through the tissues and vasculature of a patient the operator looses his or her ability to appreciate the forces restricting catheter motion, tissue properties, anatomic information and clinically important physiological signals due to the attenuation and frictional effects of intervening tissues and the compliant nature of the inserted catheters. Physiologically relevant properties of biological tissue, vascular structures and blood flow are unable to be appreciated qualitatively or measured quantitatively during percutaneous cardiac procedures.

In response to limitations of current technologies, the present inventor Dr. Stuart O. Schecter has designed specific sensor constructs, catheters/conductors and handles. These designs are capable of providing an operator with real time tactile feedback, force feedback, an operating system, and a haptic display/interface that provides physiological information, guides percutaneous cardiovascular procedures, lowers complications rates, improves outcomes, and reduces procedural times. Examples of prior works of Dr. Schecter, in the area of haptics include: U.S. Pat. No. 7,963,925, entitled “Method and Apparatus for Defining the Effect of Atrial Arrhythmias on Cardiac Performance And Directing Therapy Using a Plurality of Intrinsically and Extrinsically Derived Signals,” which discloses a catheter with a sensor and handle arrangement that provides real-time, proportional haptic feedback; U.S. Pat. Pub. No. 2012-0265076 A1, entitled “Microfabricated Cardiac Sensor with Tactile Feedback and Method and Apparatus for Calibrating the Same Using a Plurality of Signals”; U.S. Pat. Pub. No. 2012-0265083 A1, entitled “Cardiovascular Haptic Handle System”; and others.

Wallace D, et al has developed a robotic catheter manipulator that includes at least one force sensor for measuring the force applied to the working catheter by a ditherer during operation (See U.S. Pat. Pub. Nos. 20070233044 and 20070197939). Force measurements are estimated and displayed to the physician via a monitor or display. Such a design is found in ablation catheters manufactured by Hansen Medical Inc., Mountainview, Calif. Though an alarm signal can notify the operator that too high a force is applied via an audio, video or haptic signal, there is no tactile appreciation or simulation of tissue mechanics/motion or blood flow present at the distal portion of the catheter.

Companies such as Intuitive, manufacturer of the DaVinci robot, (See U.S. Pat. No. 8,004,229, Software center and highly configurable robotic systems for surgery and other uses) and Hansen Medical (See U.S. Pat. No. 7,974,681, Robotic catheter system) implement haptic feedback systems that provide notification signals and vibrotactile sensations as an alert when too much force is applied or to direct a user controlling robotic and telesurgical operative systems but are not applicable to bedside catheter based diagnostic and therapeutic procedures. These systems also do not provide the operator with tangible sensations that are physiologically relevant.

While these patents and applications provide new and novel systems and methods for minimally invasive, hand-held surgical interfaces with haptic feedback, it would be desirable to provide improvements that specifically enhance catheter balloon therapy interventions and the catheter therapy delivery components used in such procedures (e.g. U.S. Pat. No. 6,780,183).

### SUMMARY OF THE INVENTION

Embodiments of the present invention are directed to a catheter handle which functions as a hand held haptic interface including at least a balloon shaped portion for feedback and control of an interventional balloon tipped catheter. The handle may be described as an optimally configured volumetric haptic display that provides real time feedback of tissue mechanics and intra-cardiac fluid dynamics and blood flow in connection with a balloon tip catheter. The handle presents this feedback to the operator as if he or she is the actual anatomic tissue being instrumented. The technologies described herein serve to provide an operator with a haptic display that uses sensors to acquire physiologically relevant data and relay such data in analog and/or digital form, in real time, to a hand held volumetric haptic display that incorporates actuators/components that are preferably the same or similar at both the distally located end-effector and proximally located catheter handle used at the patient bedside. The data can be used to guide procedures and provide clinically relevant diagnostic information.

Embodiments of the invention include a haptic handle system for balloon-tipped catheter interventions that provides real-time haptic feedback in the form of tangible sensations to an operator. The system includes a catheter assembly including a catheter including an elongate body having a proximal end and a distal tip and an end effector, including an interventional balloon having a plurality of sensors, located proximate the distal tip of the catheter. The system further includes a haptic handle coupled to the proximal end of the catheter, where the haptic handle including a balloon-shaped haptic interface that exhibits geometric characteristics reflecting an anatomy of the interventional balloon and provides tangible sensations representative of tissues surrounding the interventional balloon. The haptic handle system further includes a control unit operably connected to the catheter assembly that acquires data from the plurality of sensors and directs actuation of the haptic interface.

Other embodiments of the invention include a haptic handle for balloon-tipped catheter interventions that provides real-time haptic feedback in the form of tangible sensations to an operator holding the handle. The haptic handle including an expandable balloon-shaped haptic interface that exhibits geometric characteristics reflecting an anatomy of a corresponding inflated balloon of a balloon-tipped catheter and sensations representative of the properties of tissue surrounding the corresponding inflated balloon. The expandable balloon-shaped haptic interface includes a plurality of actuators.

Other embodiments of the invention include a haptic handle system for balloon-tipped catheter interventions that provide real-time haptic feedback in the form of tangible sensations to an operator, including a catheter assembly and a bimanual haptic handle. The catheter assembly comprising: a catheter with an elongate body having a proximal end and a distal tip; and an interventional balloon located proximate the distal tip of the catheter. In this haptic handle system, the bimanual haptic handle is coupled to the proximal end of the catheter and includes a haptic interface that exhibits geometric characteristics reflecting an anatomy of the interventional balloon and provides tangible sensations representative of tissues surrounding the interventional balloon. The bimanual haptic handle including means to impart haptic feedback to both hands of the operator where the haptic feedback is imparted to a first more distally located section for one hand and a second more proximally located section for another hand.

Further embodiments of the invention include methods of operation for a haptic handle system. Methods include providing a haptic handle for balloon-tipped catheter interventions, including an expandable balloon-shaped haptic interface with a plurality of actuators. Methods include reproducing geometric characteristic reflecting an anatomy of a corresponding inflated balloon of a balloon-tipped catheter and sensations representative of the properties of tissue surrounding the corresponding inflated balloon with the expandable balloon-shaped haptic interface in real-time.

### BRIEF DESCRIPTION OF THE DRAWINGS

The invention may be more completely understood in consideration of the following detailed description of various embodiments of the invention in connection with the accompanying drawings, in which:

FIG. 1 shows a haptic handle with an expandable and retractable balloon portion as well as a corresponding in vivo end effector (e.g. cryoballoon, coronary angioplasty balloon) with sensor strips, according to an embodiment of the invention.

FIG. 2 shows components of a haptic handle system including: an example of pulse wave Doppler ultrasound waveforms of pulmonary venous blood flow; a balloon in vivo sensing pulsatile waveforms from pulmonary vein with a piezoelectric sensor (e.g. inverted speaker); and a haptic handle with a comparable sensor as a transmitting speaker, according to an embodiment of the invention.

FIG. 3 shows a volumetric end effector balloon based sensor and actuator with subsegmental volumes, according to an embodiment of the invention.

FIG. 3A shows a cross-sectional view of a catheter design according to an embodiment of the invention.

FIG. 3B shows a schematic cross-sectional view of the haptic interface of a handle 12, according to an embodiment of the invention.

FIG. 4 shows a sensor and actuator similar to FIG. 2, with an additional sensor depicted at the distal aspect of guiding catheter in place of the sensor being deployed on the balloon, according to an embodiment of the invention.

FIG. 5 shows a sensor and actuator similar to FIGS. 2 and 4 that illustrates carbon nanotube conductors and a detachable handle connected to a balloon shaped Haptic Handle, according to an embodiment of the invention.

FIG. 6 illustrates a tri-axial linear actuator based haptic display, according to an embodiment of the invention.

FIG. 7 shows an illustration of Meissner's corpuscle.

FIG. 8 depicts sensor and actuator technologies based on Meissner's corpuscle, nanotechnology based piezoelectric sensors and carbon nanotube conductor hybrid with conventional metal conductor, the outer shell of smart fluid or smart material such as electrorheologic and magnetorheologic fluid, high frequency vibrotactile actuators, and low frequency actuators, according to embodiments of the invention.

FIG. 9 is a flow diagram illustrating the workings of an embodiment of the invention.

FIG. 10 is a flow diagram illustrating the workings of an embodiment of the invention.

FIG. 11 illustrates the housing of actuators and motors within the handle, and specifically, a single actuator design with spokes, according to an embodiment of the invention.

FIG. 12 illustrates cross-sectional view of a knob or collar segment of a spoke support mechanism for a peripherally located haptic element, according to an embodiment of the invention.

FIG. 13 illustrates a tri-axial linear actuator based haptic display, according to an embodiment of the invention.

FIG. 14 shows a cross sectional view of a portion of a volumetric haptic handle, according to an embodiment of the invention.

FIG. 15 shows a bi-manual haptic handle, according to an embodiment of the invention.

### DETAILED DESCRIPTION OF THE DRAWINGS

The invention may be embodied in other specific forms without departing from the essential attributes thereof. The illustrated embodiments should be considered in all respects as illustrative and not restrictive.

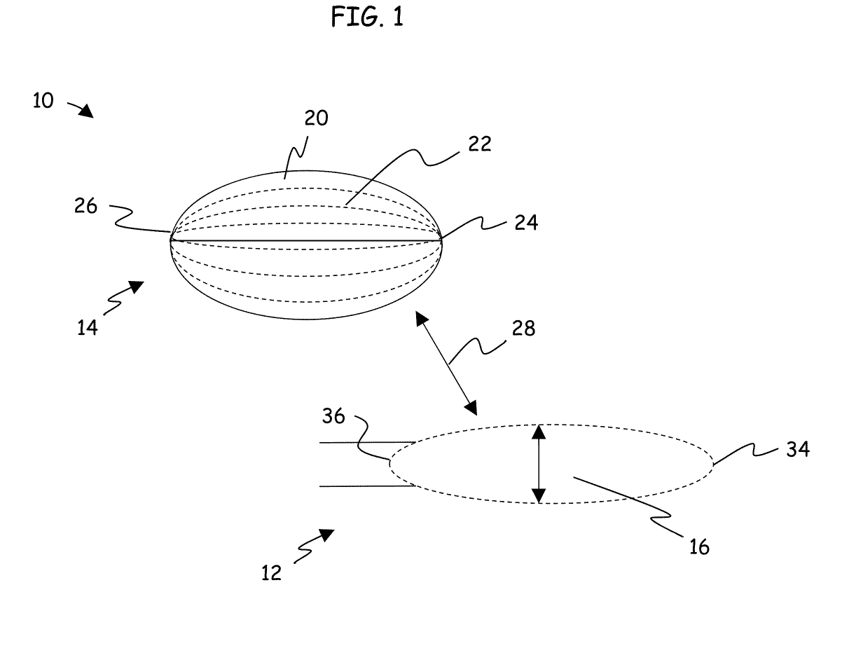

FIG. 1 illustrates one embodiment of the invention. Specifically, a haptic handle system 10 is disclosed, including a haptic handle 12 as well as an in vivo end effector 14. Haptic handle 12 is a specialized surgical handle that can be used in connection with various catheter procedures. Haptic handle 12 provides real time feedback of tissue mechanics and intra-cardiac fluid dynamics via haptic sensations delivered to an operator such that he or she feels what it would be like to be the anatomic tissue being treated in situ, in real time. The haptic handle 12 includes at least an expandable haptic interface 16. In some embodiments, the expandable haptic interface 16 of the handle 12 is generally balloon shaped. In some embodiments, this expandable haptic interface 16 may further constitute a spheroid, ellipsoid, egg shaped structure or other three dimensional shape. End effector 14 may comprise an interventional, inflatable balloon structure 20 that may be implemented within the body of a patient at the distal end of a catheter for numerous types of surgical interventions. Surgical procedures may utilize such an end effector 14 as a cryoballoon or coronary angioplasty balloon for example. The end effector 14 is generally equipped with sensor strips 22 depicted in a spaced apart manner about the perimeter of the balloon-like shape. The end effector 14 and balloon 20 may be generally described as having a proximal aspect 24 and a distal aspect 26. Similarly, the expandable haptic interface 16 of the haptic handle may be described as having a proximal aspect 34 and a distal aspect 36. Embodiments of the present invention enable the expandable haptic interface 16 of the haptic handle 12 to provide haptic feedback regarding the end effector 14 located in vivo at the distal end of a balloon tip catheter. Accordingly, actions/reactions 28 of the end effector 14 produce similar actions/reactions 28 in the haptic handle 12. Further, actions/reactions 28 on the haptic handle 12 are further capable of producing similar actions/reactions 28 in the end effector 14.

The inventor has recognized limitations, deficiencies, needs and general characteristics of past medical surgical equipment and procedures across a variety of areas to develop the current haptic handle concepts. Some of these areas are discussed in the following:

Catheters

Medical catheters and sheaths are generally tubular shaped and of a sufficiently small diameter to be inserted into a patient's body through a small incision, puncture or a natural opening. Such catheters can be used to deploy inner catheters, cardiac leads, electrodes, deliver contrast (e.g. radiopaque dye) or ablative energy in form of electromagnetic energy (e.g. current, radiofrequency energy, light), thermal energy (e.g. cryoablation) and are flexible as described by Brock et al in U.S. Pat. No. 7,931,586 and others.

Catheters for performing coronary/peripheral angiography and vascular interventions are also well understood by those experienced in the art. Catheters have been designed for engaging the coronary sinus and positioning pacing leads about the left ventricle for cardiac resynchronization therapy that is often difficult and time-consuming requiring large amounts of radiation exposure. These catheters can also dissect vessels and intracardiac structures leading to cardiovascular collapse. Unfortunately, the operator cannot appreciate the forces along the distal portion of these catheters and mainly relies on radiographic images during catheter manipulation (e.g. fluoroscopy). These images are two-dimensional and necessitate exposure to radiation. Tactile feedback systems incorporated into the design of these catheters would reduce complication rates, expedite procedures and minimize radiation exposure to the operator and patient alike.

Catheters at highest risk for causing complications include those used for atrial fibrillation ablation. Ablation of atrial fibrillation can be accomplished by delivering thermal energy to cardiac tissue, radiofrequency energy, laser energy and cold via cryoablation. Balloon tip catheters are used for cryoablation and involve expanding a balloon at the distal tip of the inserted catheter within the pulmonary venous os. Complications that can occur as a result of ablating tissue about the pulmonary veins include pulmonary venous stenosis, recurrent atrial fibrillation secondary to incomplete ablation, cardiac perforation/tamponade, diaphragm paralysis and stroke. The latter complication occurs from thromboembolism where a blot clot within the heart embolizes to the brain. The left atrial appendage (LAA) is usually where clots lodge and catheter manipulation or ablation within the LAA increases the likelihood of stroke. The LAA is proximate to the left upper pulmonary vein and catheters can be situated mistakenly in the LAA rather than the pulmonary vein. The pulmonary vein and LAA have characteristic tissue motion and blood flow properties that can be differentiated with tactile feedback as described in more detail below.

Recent studies have found cryoablation to be significantly more effective than medication, and patients generally experience less pain than with radiofrequency ablation. Using cold, rather than heat, to disable damaged tissue reduces the chances of impacting healthy heart tissue and surrounding structures. To perform cryoablation, a doctor inserts the balloon catheter into a blood vessel, usually in the upper leg, and then threads it though the body until it reaches the heart. This narrow tube has an inflatable balloon on one end that engages the pulmonary vein. Using advanced imaging techniques, the doctor is able to guide the catheter to the heart but imaging does not provide three dimensional confirmation of anatomic location or provide the operator with feedback about catheter tissue contact or tangible sensations characteristic of anatomic location and cardiac function.

The specific application of a tactile feedback system to such catheter designs will enable the operator to palpate tangible sensations due to blood flow and tissue mechanics thereby improving procedural success and reducing complications. Though the technologies discussed in this disclosure focus on the application of this technology for pulmonary vein ablation using cryoablation techniques, they can be applied to a variety of procedures using balloon tipped catheters within the heart and vasculature (e.g. engaging the coronary arteries and stent deployment, coronary sinus, and performing peripheral vascular interventions).

Handles

Conventional handles at the proximal end of an inserted catheter allow the operator to manipulate inserted catheters. Handles are used for positioning pacemaker leads, catheters, or intravascular delivery/extraction systems, integrated into ablation catheter systems and the like.

Sensors

Miniaturized sensors such as piezoelectric sensors or accelerometers are under development and hold promise to acquire intra-cardiac data representative of myocardial wall motion. Accelerometers can be placed along the body of the catheter and distally about electrodes or implanted leads positioned to be juxtaposed to cardiac ventricular wall locations, such as the left ventricle free wall, right ventricle free wall, and the anterior/septal/lateral wall or other intra-cardiac (e.g. endovascular) and extra-cardiac surfaces (e.g. epicardial). The accelerometers produce signals in response to the motion of the ventricular wall locations that relate to mechanical tissue characteristics during the cardiac cycle. An example of how this technology can be applied to programming of timing intervals within an implanted CRM device can be found in U.S. Pat. No. 7,127,289 by Yinghong Yu, which is hereby incorporated by reference herein.

Aeby and Leo invented tri-axial force sensors that use optical fibers to generate variable intensities of light as a function of deformation See U.S. Pat. Pub. No. 20080009750, which is hereby incorporated by reference herein. This system provides the operator with measurements of contact forces at the catheter's distal end but does not provide tactile feedback simulating cardiac mechanics.

Advances in nanotechnology have enabled fabrication of discrete carbon nanotube based nano-scale force sensors with specific spatial orientations using electron beam lithography (Stampfer, C.; Jungen, A.; Hierold, C. Sensors, 2004. Proceedings of IEEE. Volume, Issue, 24-27 Oct. 2004 Page(s): 1056-1059 vol. 2). Singh et al. (Nanotechnology 2007 18 475501) demonstrate the design and calibration of an individual carbon nanotube (CNT) based mechanical force sensor for measuring cell wall compliance using a micromanipulator inside a scanning electron microscope. These micromachined piezoelectric sensors can calculate the bending modulus of individual multi-walled carbon nanotubes. Ezhilvalavan S, et al. fabricated freestanding lead zirconate titanate force sensors as a parallel plate capacitor structure in various shapes and dimensions (J. Phys 2006.: Conf. Ser. 34 979-984). Kim, Jong-ho developed piezoelectric force sensors (See U.S. Pat. Pub. No. 20050262676) that detect contact pressure along vertical and horizontal axes using oxide metal films and in U.S. Pat. Pub. No. 20060059997. U.S. Pat. Pub. Nos. 20050262676 and 20060059997 are hereby incorporated by reference herein. Kim, Jong-Ho et al also designed a wiring structure for a tri-axial piezo-resistive force sensor placed in multiple directions to sense a contact force with respect to X and Y axes in parallel with a substrate and the Z axis perpendicular to the substrate. MEMS based sensors can also be utilized. Wireless piezoelectric sensors using resonant radiofrequency cavities convert mechanical properties of attached structures into electrical signal changes (Chuang J eta 1. Embeddable wireless strain sensor based on resonant RF cavities. Rev. Sci. Instrum., vol 76, no. 9, p 094703, 2005). These sensors can be used to acquire signals at and about the end-effector of inserted cardiac instrumentation. Wireless displacement sensors based on reverse design of microwave and millimeter-wave antenna arrays have recently developed for structural monitoring purposes (Rizzoli V, et al. A New Wireless Displacement Sensor Based on Reverse Design of Microwave and Millimeter-Wave antenna Array. IEEE Sensors Journal, Vol. 9, No. 11, November 2009. p 1557).

Haptics

Currently available haptic displays include but are not limited to programmable keyboards, augmented mice, trackballs, joysticks, multi-dimensional point and probe-based interactions, exoskeletons, vibro-tactor arrays, gloves, magnetic levitation, and isometric devices (Burdea, G C. Force and Touch Feedback for Virtual Reality. New York: Wiley Interscience, 1996).

Simplified tactile virtual reality interface devices are known by those experienced in the art and have been used in game controllers. These interface devices are becoming more complex as to better simulate sensations such as hitting a tennis racket. See the Handheld computer interface with Haptic Feedback of U.S. Pat. Pub. No. 20110121953 to Grant et al, which is hereby incorporated by reference herein. Haptic technology has been used to simulate medical procedures, for example, for teaching purposes. See U.S. Pat. Nos. 5,389,865 and 5,769,640 to Jacobus et al., which are each hereby incorporated by reference herein.

Cryoablation Systems

During cryoablation for treatment of atrial fibrillation a balloon is positioned at the ostium of the pulmonary vein, and extreme cold energy flows through the catheter to destroy this small amount of tissue and restore normal sinus rhythm. Examples of balloon based catheters include the Arctic Front® Cardiac CryoAblation system (Medtronic CryoCath LP) and the Cardiofocus Endoscopic Ablation System.

The Artic Front CryoAblation system is engineered with a balloon-within-a-balloon design. Liquid nitrous oxide is passed within the inner balloon. A constant vacuum is applied between the balloons to prevent any leakage of refrigerant should the inner balloon be defective. The cryoballoon is available in 23 mm and 28 mm sizes in order to accommodate varying pulmonary vein anatomy. The balloon is advanced over a 0.035 inch wire via the 12 Fr FlexCath® unidirectional steerable sheath. The balloon itself has 45 degrees of bidirectional movement. Any tissue in contact with the balloon freezes with tissue temperatures reaching below minus 50° C. as the liquid nitrous oxide expands to gas. The freeze renders the tissue electrically inert. As a result, the pulmonary veins become electrically isolated when balloon cryothermal ablation is applied to the antrum of the pulmonary veins. This system allows monitoring of pulmonary vein electrical activity so the operator can better identify when complete electrical isolation of the pulmonary vein has been achieved, though recurrent atrial arrhythmia remains frequent with currently available technology. The latter is accomplished with the Achieve Mapping Catheter which has a 15-20 mm mapping diameter and 8 electrodes and is delivered through the CryoBalloon during ablation procedures.

The Cardiofocus Endoscopic Ablation System includes a disposable, 12F, multi-lumen catheter which has an inflatable balloon at its distal end made of a compliant material and is specially shaped to provide wide areas of contact within the pulmonary vein ostium. The balloon size is dynamic as a function of the pressure used to provide inflation. It can span from 20 mm to over 30 mm in diameter. An Arc Generator laser delivery fiber, included with the catheter, provides the conduit to deliver a 30-degree arc of laser energy. The Arc Generator is advanced through the central lumen of the Balloon Catheter for energy delivery. The fluid within the balloon is used to cool the arc generator and passes through the central lumen of the catheter. Separate lumens are available for balloon inflation/deflation, endoscope access and illumination. The system is delivered through a 12 French deflectable sheath introducer that performs transeptal puncture and accesses each of the four pulmonary veins and has a 500 micron, re-usable endoscope for visualization during positioning of the catheter and balloon.

Benefit of Haptic Feedback

Central nervous system processing of tactile stimuli and reaction times to tactile information are faster than to visual stimuli and the presence of haptic feedback improves attention, cognition and performance. The Multiple Resource Theory of attention explains that people have separate pools of attentional resources for the different sensory modalities and that tactile displays are particularly advantageous in situations where auditory and visual channels are heavily loaded, providing faster reaction times than visual feedback alone, especially for alerting operators to unexpected, high priority events and as a redundant cue to visual alerts. With haptic feedback, the operator will not need to visualize another display in addition to focusing on radiographic images, intracardiac electrical data, hemodynamic monitoring, and 3D navigational images. See United States Army Research Laboratory: A Review and Meta Analysis of Vibrotactile and Visual Information Displays, September 2009.

No conventionally available ablation catheters provide the operator with real time reproduction of tissue motion or blood flow. It is recognized that the ability to appreciate intra-cardiac tissue mechanics, blood flow and other physiologically relevant properties will improve procedural success, reduce procedural times, and limit risk of complications such as pulmonary venous stenosis and cardiac perforation. Embodiments of the present invention include a hand held volumetric haptic display that reproduces the physical events occurring at the catheter's distal aspect (e.g. proximate to or about the balloon tip) for the operator at the handle or proximal end of the catheter. The system acquires real time diagnostic information about the vasculature (e.g. pulmonary venous anatomy, coronary artery properties), tissue mechanics, the surrounding structures (e.g. LAA), and the fluid dynamics of intracardiac and intravascular blood flow. In one embodiment, the system is used in conjunction with a catheter based system with one or more expandable balloons at its distal end used for treatment of obstructive vascular lesions or the delivery of therapeutic energy or medications, as described in detail below.

Pulmonary Vein and Pulmonary Venous Blood Flow

Though embodiments of this invention are primarily described in the context of ablation procedures for the treatment of atrial arrhythmias (e.g., pulmonary vein isolation), they are in no way limited in scope and spirit and are applicable to intravascular and intracardiac procedures, transcutaneous aortic valve replacement, valvuloplasty, mitral valvular repair/replacement, coronary and endovascular angioplasty/stent deployment, etc.

The four pulmonary veins are large blood vessels that carry blood from the lungs to the left atrium. They carry oxygenated blood to the heart prior to delivery of the oxygenated blood to the body's tissues. Equipment designed to perform procedures for curing atrial fibrillation aim to ablate the electrical pathways between the pulmonary vein and left atrium. Ablation procedures for atrial fibrillation are being performed with increasing frequency with patients often having two or more repeat procedures. One of the most serious complications is the development of pulmonary vein stenosis. The presentation of pulmonary vein stenosis varies widely. The majority of patients are symptomatic although specific referral bias patterns can affect this. Symptoms may include dyspnea or hemoptysis or may be consistent with bronchitis. Progression of stenosis is unpredictable and may be rapid, (Pulmonary Vein Stenosis Complicating Ablation for Atrial Fibrillation: Clinical Spectrum and Interventional Considerations. David R. Holmes, JrJ Am Coll Cardiol Intv, 2009; 2:267-276) with dramatic increases in pulmonary venous inflow resulting (Tabata, T. et al. J Am Coll Cardiol, 2003; 41:1243-1250).

Pulmonary vein narrowing demonstrated on cardiac MRI has been found to occur in 24-38% of patients having undergone just one AF ablation procedure (Pulmonary Vein Anatomy in Patients Undergoing Catheter Ablation of Atrial Fibrillation: Lessons Learned by Use of Magnetic Resonance Imaging. Kato R et al. Circulation. 2003; 107: 2004-2010 and Incidence and Predictors of Pulmonary Vein Stenosis Following Catheter Ablation of Atrial Fibrillation using the Anatomic Pulmnoary Vein Ablation Approach: Results from Paired Magnetic Resonance Imaging. Dong J et al. J Cardiovasc Electrophysiol 2005, Vol 16, pp. 845-852). Progression of pulmonary vein stenosis can occur over time (Dill T et al. Circulation 2003; 107, 845-850) and can lead to chronic pulmonary conditions, persistent cough, hemoptysis, pleural effusions, congestive heart failure and pulmonary hypertension. Pulmonary vein ostium shape and orientation has been shown to be predictive of occlusion in patients with drug-refractory paroxysmal atrial fibrillation undergoing cryoballoon ablation (“CBA”); (Sorgente, A. et al. Europace (2011) 13 (2): 205-212). CBA techniques may suffer from an inability to create an adequate seal within varying atrial/pulmonary venous geometric anatomy and incomplete ablation. Phrenic Nerve Palsy resulting from inappropriate cryoballoon to PV diameter leading to undersized balloons being positioned too deeply within the PV (Chun KR et al. J Cardiovasc Electrophysiol. 2009; 20(11)1203-1210), while oversizing the balloon results in distortion of the PV orifice and shorter distances between the cryoballoon and phrenic nerve with resulting impingement (Sarabanda A V et al. JACC 2005; 46(10):1902-1912).

The frequency of pulmonary vein stenosis is expected to increase in patients undergoing more than one ablation procedure. Serial evaluation of the pulmonary veins and atrial mechanical function is possible with haptic based monitoring. Pulmonary venous stenosis results in increases in blood flow velocity and turbulence (Pulmonary venous flow by doppler echocardiography: revisited 12 years later. Tomotsugu T et al. J Am Coll Cardiol, 2003; 41:1243-1250, Robbins I M, Colvin E V, Doyle T P, et al. Pulmonary vein stenosis after catheter ablation of atrial fibrillation. Circulation. 1998; 98:1769-1775). This can be appreciated qualitatively and quantitatively with the haptic technology described herein as is discussed in more detail below. This technology will also enable the clinician to derive information about intracardiac anatomy (e.g. based on the shape of an inflated cryoballoon) which can be appreciated tactually and characterized by an operator holding the haptic handle, as described in more detail below.

Atrial Mechanics

Though ablation of atrial fibrillation is aimed at improving clinical symptoms, and in theory, improving cardiac function and atrial mechanical characteristics, data is emerging that scarring after ablation can affect LA transport and systolic function and lead to progressive dyspnea even in the absence of pulmonary vein stenosis. This Stiff Left Atrial Syndrome, characterized by pulmonary hypertension, has recently found to be more often diagnosed in post-ablation patients despite persistent sinus rhythm (Gibson D N et al. Stiff left atrial syndrome after catheter ablation for atrial fibrillation: Clinical characterization, prevalence and predictors. Heart Rhythm, Vol 8, No. 9, 2011). The authors diagnosed this syndrome of diastolic LA dysfunction based on invasive hemodynamic measurements using right heart catheterization (echocardiographic techniques were non-diagnostic). Regional mechanical abnormalities have also been noted along the roof of LA based on CT imaging in four dimensions in a separate study (Tsao H M et al. J Cardiovasc Electrophysiol 2010; 21: 270-277). In a recently published study stroke risk was found to be associated with ablation of atrial fibrillation after radiofrequency and cryoablation MAZE procedures. Patients at highest risk were found to have impairments in atrial mechanical function and increased atrial dimensions (Buber J. et al. J Am Coll Cardiol, 2011; 58:1614-1621). Of note, patients with impairments in atrial mechanical function, despite being in normal sinus rhythm, had a five fold increase in thromboembolic stroke at mean follow-up of two years (n=150).

The ability to measure and qualitatively appreciate left atrial mechanical function and pulmonary venous inflow before, during and after ablation procedures with the technologies described herein will provide the operator with a means for identifying physiologic indices that herald the development of pulmonary venous stenosis and Stiff Left Atrial Syndrome. A means for digitizing and storing acquired data during ablation procedures will enable objective assessment of acquired data, especially for patients undergoing multiple ablation procedures who are at higher risk for such complications. As more patients are expected to undergo repeat procedures and the total number of patients suffering from complications due to ablative techniques increase, there will be an increased need to identify sub-clinical findings that preempt clinically significant consequences.

Sensor Design

In order to accurately acquire the most relevant physiologic data during balloon cryoablation, embodiments of the present invention include sensor systems that utilize nanotechnology (e.g. circumferentially positioned nanosensors). In one embodiment, more than one sensor type (e.g. force sensors) and multiple sensors are used to gather real time physiologic information such as the contact force between the balloon and tissue (e.g. endothelium at the pulmonary venous os, intravascular tissue during angioplasty procedures), deformation of the balloon, tissue motion and blood flow (e.g. identifying pulmonary venous stenosis, intra-coronary stenosis) for representation in the haptic display.

Embodiments of the present invention are not limited in sensor design and other types of sensors including but not limited to electromagnetic systems (e.g. fiberoptics), conventional piezoelectric materials, miniaturized strain gauges, electrically based sensing systems (e.g., resistive, impedance, current, voltage, admittance, etc.), and shape-memory-alloys can be used to gather information about tissue mechanics and multiple actuator types used to provide haptic feedback. Nor is its application limited in scope and spirit as it applies to other interventional procedures (angioplasty, stent deployment, endovascular procedures etc.) where haptic feedback will provide the operator with a tactile appreciation of physiological and anatomic properties from a novel vantage point. Embodiments of the present invention enable the operator to feel what it would be like to be the anatomic tissue being treated in situ, in real time.

Referring to FIG. 1, sensor strips 22 (e.g. ribbons of elastic piezoelectric material such as ZnOxide, or miniaturized strain gauges) course along or are positioned about the surface of one or more components of the inflatable balloon 20 of the end effector 14. These strips 22 can be oriented in any number of ways including helically, circumferentially and/or longitudinally arranged configurations. The sensor strips 22 deform and stretch, ideally in similar fashion to the inflated balloon 20, and a voltage is generated from such deformation. The sensor strip transducer is ideally low profile, less than 0.5 mm thick with surface areas between 0.25-5.0 mm², optimally designed to have the appropriate Young's modulus, permittivity, dielectric, piezoelectric and elastic constants for acquiring relevant physiological signals and when necessary will be able to perform under inclement conditions, such as during cryoablation where temperatures are often below −50 degrees C. The strain gauges are likewise of low profile, with a surface area and thickness that allows multiple sensors to be positioned in a fashion (e.g., with enough distance between sensors) to provide regional information. The sensor construct and/or system processing account(s) for the temperature dependence of the material properties of the transducer material chosen (Yang, SSG et al. Temperature Dependence of the Dielectric, Elastic, Piezoelectric Material Constants of Lead Zirconate Titanate Ceramics mastersonics.com/documents/mmm_basics/general . . . /ferro29.pdf).

Each of multiple sensors or sensor strips 22 are deployed in specific regions of one or more segments or sections of an inserted catheter and/or its attachments. These acquire data in large part from regions of interest that are assigned to specific, associated regions of the hand held haptic display or interface of the haptic handle 12. Thus, the operator perceives tangible haptic sensations associated with locales and appreciates regional information. By way of example, during deployment of intravascular stents or prosthetic heart valves, cryotherapy of cardiac tissue with inflatable balloons, valvuloplasty and the like, sensors located in areas where there is inadequate contact between the delivery mechanism (e.g. inflatable balloon) and/or deployed therapeutic device and the tissue undergoing treatment will generate a different signal (e.g. piezoelectric voltage) due to less force, deformation, displacement than neighboring regions of interest. This is palpable at the haptic interface 16 (e.g., balloon shaped or ellipsoid handle portion). In one embodiment, the operator can adjust force manually (e.g., pneumatic, hydraulic, electric), automatically or robotically in these regions by selectively controlling the pressure or force generated along different anatomic segments via one or more controllers on the haptic interface 16 or in a separate location. This is of particular benefit when the treated areas have anisotropic anatomic (i.e. geometric) or physical properties (e.g, elasticity, rigidity). For example, the pulmonary venous os may be shaped in a non-circular fashion and require cryoablation balloons to exert different degrees of atmospheric pressure in neighboring regions in order to effectively ablate tissue, and certain stenotic vessels or cardiac valvular tissue may require greater expansion due to calcified intravascular plaque along a portion of the circumference of the treated vessel or valve. Certain segments will require more pressure during stent or prosthetic valve deployment in order to avoid future restenosis, or regurgitation of blood, respectively. It is not mandatory for the user to know the precise anatomic location the tangible sensations are associated with, though in one embodiment of the invention, external positioning systems or navigational systems (e.g., electric, magnetic) could be used to localize specific segments of the inserted treating member. In one embodiment, blood flow properties are appreciated. For example, laminarity, turbulence, regional regurgitant blood flow jets are palpable.

In one embodiment, the sensor(s) is/are situated between two or more layers of material (i.e. sandwiched between two balloons) or one layer (or balloon) is composed of sensor material (e.g. piezoelectric material/composite) or, by way of example, disposed of as segments located at specific locations about or within the balloon that has a length of between 5 and 15 mm.

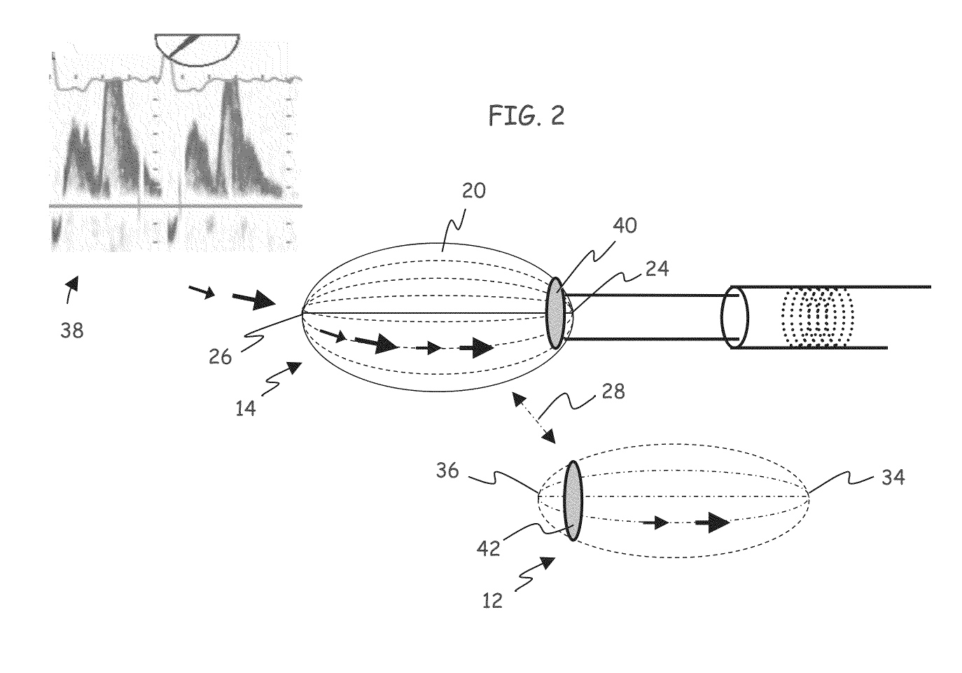

FIG. 2 depicts an alternate embodiment, in which a sensor 40 is located at the proximal aspect 24 of the balloon 20, functioning as an inverted speaker that receives the appropriate physiological signals indirectly. Likewise, an actuator 42, similar in construct to the sensor 40 (e.g., piezoactuator), is positioned at the distal aspect 36 of the haptic handle 12 and generates the appropriate signals to actuators within or about the handle 12. Preferably, the medium that conducts and transmits the physiologic signals (e.g. vibration) to the operator within the haptic handle 12 has properties (e.g. acoustic) that ensure that the velocity of the propagated signal and palpable acceleration/deceleration (e.g., due to the moving shaft of a linear actuator) are much the same as the imparted motion to the sensor(s) 40 at the level of the end-effector 14. Thus, pulsatile blood flow from within the pulmonary veins, for example, will have a specific velocity range (e.g. 5-50 cm/sec) and this will be palpably reproduced for the operator. Sensor design considerations will ensure a faithful, non-virtual recreation of physiological events (e.g. higher velocity due to pulmonary venous stenosis, turbulent blood flow due to microbubble formation during ablation of cardiac tissue, non-laminar non-uniform regurgitant blood flow from valvular pathology). An example 38 of Doppler ultrasound waveforms of pulmonary venous blood flow is shown in FIG. 2.

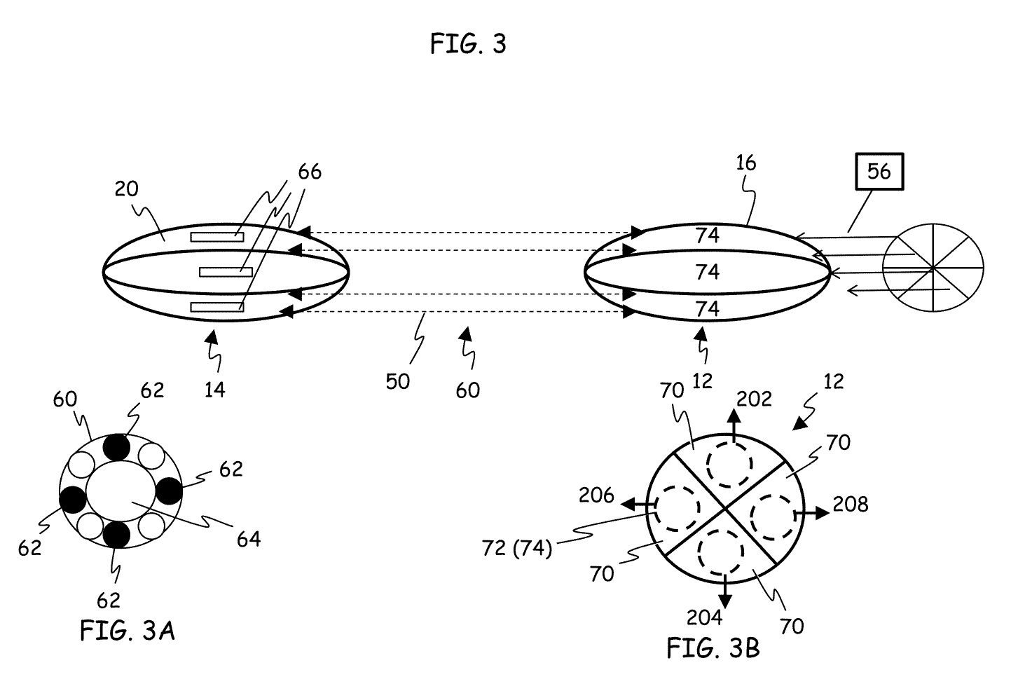

FIG. 3 depicts one embodiment of the invention where the medium used to inflate the balloon mechanism 20 of an end effector 14 to deliver therapy (e.g., deploy stents/prosthetic valves, perform valvuloplasty, cryotherapy) consists of the actual liquid or gaseous substance (e.g., perflouorocarbon) that is in communication or fluidly coupled (as represented by arrows 50) to at least one lumen along the catheter shaft 60, one or more portions of the treating balloon 20, and subsegmental volume depots positioned about the hand held haptic interface 16 of haptic handle 12 designed to provide tangible sensations. By way of example, delivery of cryotherapy and balloon expansion (e.g., 5-50 atmosphere) can occur by a variety of mechanisms as known by those experienced in the art, including activation of a gear pump 56 that circulates the medium into multiple tubes 62 that course within the inserted catheter 60 (e.g. 6-16 French) through individual supply lumens which are fluidly connected. The catheter 60 is constructed of material such as polyether blockamide, polyamides, and polyurethanes, that can be divided up with membranes of material or multiple conduits 62 which course along the peripheral portion of the catheter body 60. This is shown in a cross-sectional view of the catheter 60 in FIG. 3A as a circular honeycomb with multiple conduits or multiple tubes 62. An inner conduit 64 (e.g., 2-14 French) houses inner wires, catheters and the like. Sensors 66 are localized along different portions of the balloon/treating member 20 and acquire regional data for presentation at the haptic interface 16 of the handle 12 at different sections 70. A schematic cross-sectional view of the haptic interface of handle 12 is shown in FIG. 3B. These sections 70 can be located about four quadrants as depicted, each with independent haptic displays. Any mechanism for delivering tangible sensations at the haptic display(s) can be used within embodiments of the present invention and include implementation of circular resonators 72, a haptic membrane composed of or in contact with electroactive polymers (EAP) 74, motors, piezoactuators, and the like, or effects due to the circulation of liquid or gaseous medium at the handle 12.

In another embodiment, wires deployed via the catheter assembly are capable of sensing tissue motion and/or blood flow as known as those experienced in the art (e.g., Doppler flowires). These wires can serve the purpose of acquiring data and guiding the catheter assembly into the correct anatomic position (e.g., within the coronary sinus and its branches, pulmonary venous os, coronary arteries, peripheral vasculature, etc.) and measure physiologically relevant signals such as blood flow velocity outside of an inserted catheter and within an inserted catheter (Guiding Catheters with Side Holes Relieve Pressure Damping and Improve Coronary Blood Flow: Assessment with the Doppler Flowire. Schecter et. al. Circulation 1994; 90: 4, part 2: I-164.) This physiologically relevant data is then recreated in real time at the level of the hand held haptic interface using haptic feedback.

In another embodiment, multiple microfabricated sensors (e.g. MEMS based or nanosensors) are located about the inflatable balloon and “innervate” multiple conductors and transducers at the operator's haptic handle 12. The advantage of this construct is that such a sensor design enables information about contact uniformity to be palpable. By way of example, multiple miniaturized strain gauges or four ZnOxide nanoribbon constructs or arrays of nanowires (Hansoo Kim and Wolfgang Sigmund. Zinc oxide nanowires on carbon nanotubes. Appl. Phys. Lett. 81, 2085 (2002)) are positioned 90 degrees apart along the long axis of the balloon 20. Each separate sensor construct acquires motion data and generates a current signal along multiple (e.g. four) separate conductors (e.g. carbon nanotube conductors (CNTC), hybrid conductors). Each conductor activates a different actuator in the haptic handle 12 and thus imparts a reciprocating haptic effect (e.g., vibrotactile and/or force signal) representative of regional motion information along each of the individual segments (e.g., four quadrants 70) of the haptic handle 12, as shown in FIG. 3B. Any number of segments 70 can be sensed/actuated for more accurate data acquisition and haptic display. Balloon inflation is palpable in such an application and regional anatomic and physiological properties (e.g. intracardiac geometry, venous inflow properties of direction of flow, flow velocity, turbulence, laminarity) are sensed by the user in real time as if the hand is the anatomic tissue receiving treatment. During cryoballoon ablation, this will enable the user to feel as if his or her hand were actually being treated by the balloon, palpating expansion, pressure at the tissue-balloon interface, pulmonary venous blood flow, left atrial appendage motion, etc.

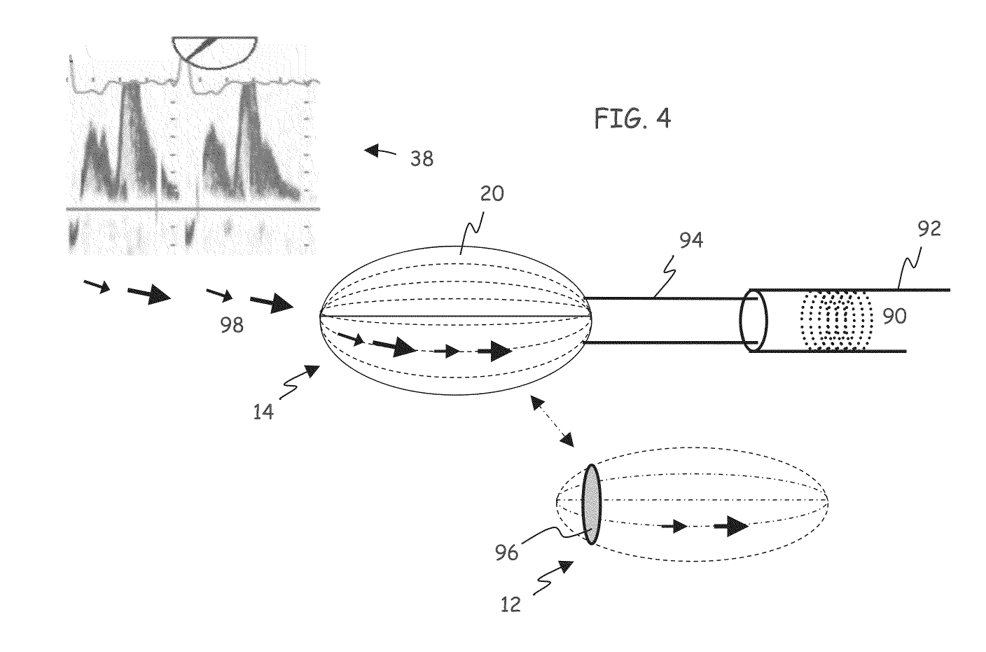

Referring now to FIG. 4, in order to avoid the affect of varying temperature on sensor performance (e.g. during cryoablation), the sensor 90 can be located such that it receives transmitted physiological signals (e.g. PV blood flow, atrial appendage motion) from conducted or transmitted vibration between the inflated balloon 20 and guiding catheter 92 where the sensor 90 is located. By way of example, the sensor 90 can be positioned at the distal aspect of a guiding catheter 92 used to direct and locate the balloon tipped catheter 94 (e.g. cryoablation instrument) within the heart or vasculature. Preferably, the system provides haptic feedback during key procedural events such as the crossing of the interatrial septum. In one embodiment, piezoelectric material is applied or painted on one or more locations of the catheter (e.g. at 90 upon conductive silver paint). A conductor is attached to the underlying conductive coating (e.g., silver) and carries current to an amplifier for generation of haptic signals to one or more actuators 96 (e.g., piezoelectric traveling wave motor) within the haptic handle 12. Pulmonary venous inflow, as sensed by ultrasonic Doppler is seen in the example 38 the top left portion of FIG. 4 and depicted as arrows of varying dimensions 98 at the end-effector 14 and haptic handle 12 and, is an example of physiological information that can be acquired and communicated with haptic feedback. Other physiological information can also be palpable including but not limited to sensing of the interatrial septum during transeptal puncture, traversing various tissue planes, perforating tissue boundaries, expanding stenotic tissue or heart valves, deploying prosthetic heart valves, tightening annular rings used to treat valvular regurgitation and mechanisms used to expand blood vessels all of which are within the scope and spirit of embodiments of the invention, which is not limited to the tactile perception of any specific anatomic or physiological phenomena. Sensor type is not intended to be limiting and may include sensors such as force sensors, pressure sensors, manometers, strain gauges, piezoelectric sensors, non-contact sensors, and others.

In yet another embodiment, balloon inflation occurs in response to the operator squeezing the haptic handle 12 or segments of the handle as to modify the regional delivery of pressure along specific portions of the balloon 20 that may require more or less inflation due to anisotropic geometry or variable tissue properties. This can occur with a variety of mechanisms including electronic, hydraulic, pneumatic systems and when a haptic interface is present, it can be accomplished manually by the user's response to palpable sensations at the handle 12 that are representative of real time events. Forces and vibrations are reciprocal between the in situ end-effector 14 and hand held haptic handle 12. Multiple sensor technologies can be utilized to acquire force and vibration data at both the end effector 14 and haptic handle 12 as discussed in more detail below.

Actuating Sensor Design

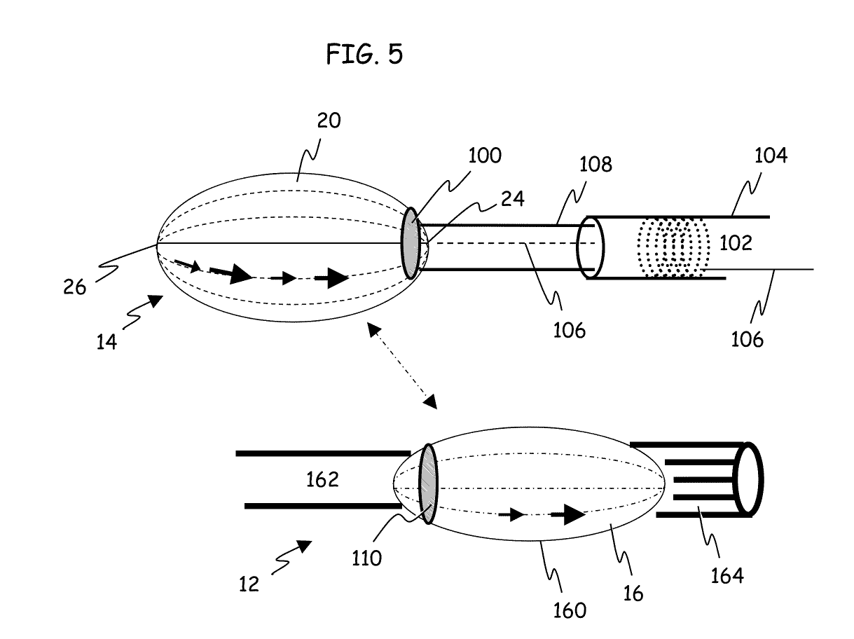

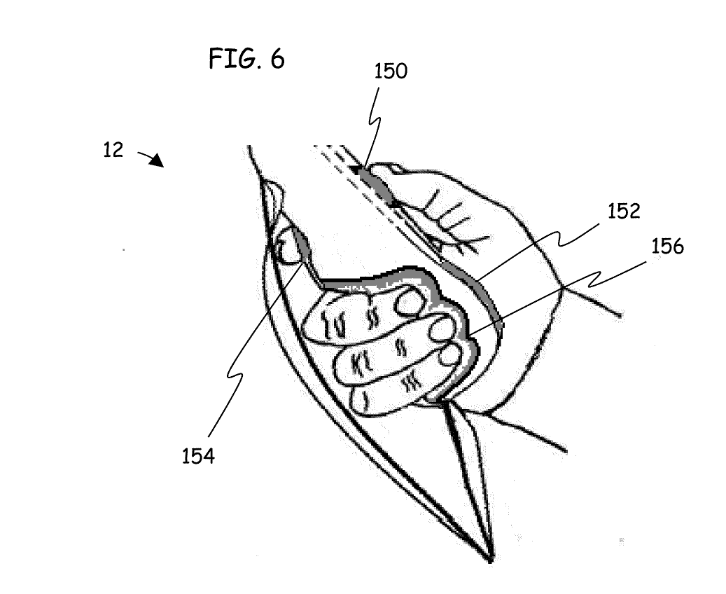

Referring to FIG. 5, the sensor is depicted as either a transmitting piezoelectric type sensor 100 (sensing speaker, piezoactuator) located at the proximal aspect 24 of the inflatable balloon 20 and/or as embedded piezoelectric material 102 at the distal aspect of the guiding catheter 104 which acquires reflected and conducted motion and flow data, respectively, which is then transmitted along a conductor 106 running (e.g., CNTC) in parallel within the body of an isodiametric catheter 108 innervating one or more piezoelectric type actuators 110 within the handle 12 (e.g. transmitting speaker, piezoactuator). Maintaining the signal in an analog form will circumvent problems associated with sampling, digitization, asymmetric dynamics, time delays due to processing, system instability due to lack of passivity. Any type of actuator within the handle 12 is within the scope and spirit of various embodiments of the invention. One preferred embodiment which incorporates sensors and actuators that have similar properties (reciprocating) and conductors that are made using carbon nanotubes will enable transmission of sensor data from multiple locations within conventionally sized catheters.

Actuators that utilize the piezoelectric effect have very fast reaction times and high dynamics as well as durability. Examples of actuators that can be implemented for generating tangible sensations representative of intra-atrial and pulmonary venous anatomy, blood flow and physiology include linear actuators, piezotronics, piezoactuators, stacked actuators, ending actuators, ultrasonic actuators, uchinomotors, stepper motors (Kern TA. Engineering Haptic Devices. (Ed.) 2009, XXXI, 472 p. 243-276). Though specific piezoelectric actuators are discussed in detail herein, embodiments of the present invention are not limited to any specific type of actuator and alternate actuators (e.g. electrostatic, electrodynamic, electromagnetic, magnetic, magnetorheologic, electrorheologic, linear actuators, rotary motors).

In one preferred embodiment, the actuator and sensor/transducer are both piezoactuators possessing high degree of precision, high bandwidth, fast response times, and submicron precision. As an actuator, these piezoactuators function by the inverse piezo effect and deform when a voltage is applied (e.g., DuraAct patches, Physik Instrumente). This contraction/deformation is controlled by affixing the actuator to a substrate material that confines and directs the deformation to conform to certain geometric shapes (profile or shape control). For example, the piezoactuator is affixed in between or underneath elastic thermoplastic polymer inlays or similar material which is positioned along key portions of a hand held handle as depicted in FIG. 6. A first piezoactuator 150 is located under the thumb of the operator, a second piezoactuator 152 is located under the thenar eminence, a third piezoactuator 154 is located under the second digit, and additional piezoactuator(s) 156 are located under the fingertips of the third through fifth digits. The reciprocating design, of having the transducer and actuator composed of the same material, optimizes system functionality by providing for a linear relationship between the sensor and haptic components. The material properties of the piezoactuators may vary, but, by way of example, the actuator/transducer can have variable dimensions but be between 0.1-0.4 mm thick, have al 5 nF capacitance, a supply voltage range between −50-200 volts, a blocking force 50-100 Newtons, and a Young's modulus between 10-50 Gpa).

In one embodiment, the handle 12 is comprised in part with an outer material balloon segment 160 circumferentially positioned about the handle exterior constructed of composite material that has similar properties to cardiac tissue and vasculature. The texture of the material (rubber, silicone, ridged, etc.) may be fabricated as to enable the operator to feel as if his or her hand is the actual anatomic tissue being treated. In one application of an embodiment of the invention, the inside of the outer material balloon segment 160 has acoustically conductive material or gel. Smart fluids and/or smart materials can be utilized to accomplish this as described in more detail below. In one embodiment, the balloon-like haptic interface 16 of the handle 12, is attached to a segment 162 that enables the catheter to be detachable from the inner components/actuators, so the haptic handle 12 can undergo sterilization and be separated from a disposable outer exoskeleton/catheter construct. Thus, in this embodiment, the main working components of the handle 12 are reusable and need not be sterilized. An outer sheath or glove can encase the handle 12 if needed for additional protection from blood and body fluids and to maintain sterility if needed. The detachment mechanism is not limited to any particular construct.

In one embodiment, a multi-fingered haptic glove and/or outer envelope with additional actuators encasing the user's hand is implemented to extend the properties of the haptic display. By way of example, such a construct will enable the operator to have a sense of enclosure or experience compressive forces along the outer surface of the hand and in between the digits.

The tail 164 of the handle 12 in yet another embodiment, contains any needed hardware, firmware or software and can be detachable. Alternatively, the control unit can be in a separate location wired or wirelessly connected to the haptic handle 12. In an alternate embodiment, the tail 164 of the handle 12 is fitted with actuators, position sensors, gyroscopes and the like to provide additional haptic and/or force feedback, and to counteract geodesic forces thereby rendering the haptic handle 12 virtually weightless, while monitoring three dimensional handle position in real time.

In yet another embodiment, the handle 12 can be attached or an integral part of a force feedback tele-robotic interventional system and provide more detailed and subtle tactile/haptic feedback.

Volumetric Reciprocating Haptic Handle

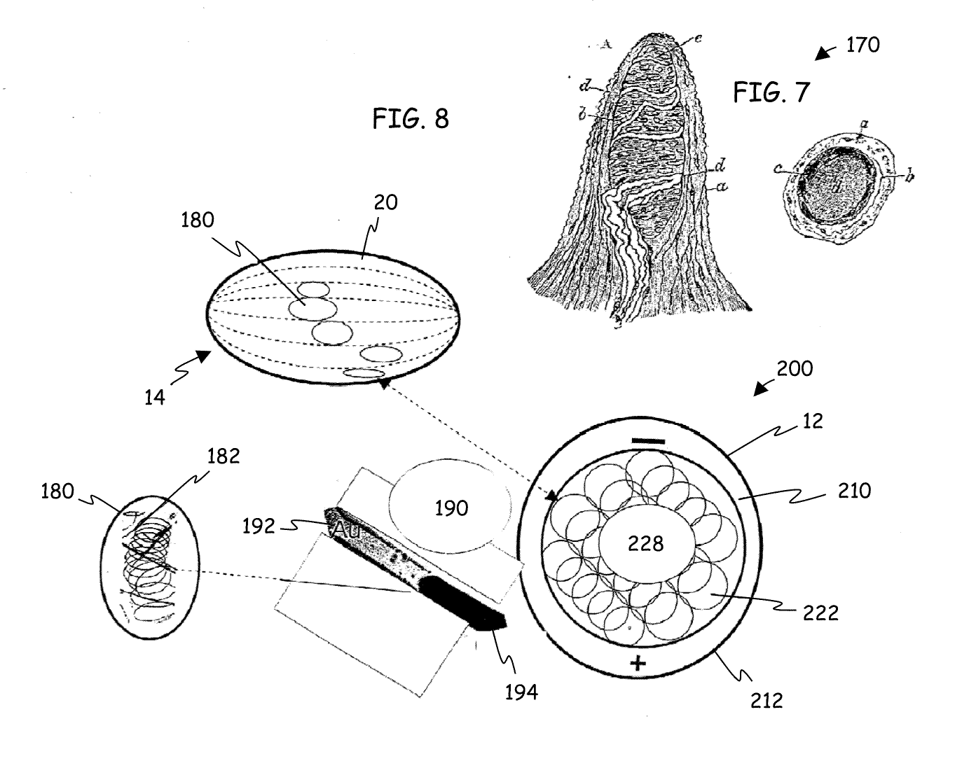

Haptic effects used to recreate the feel of moving biological tissue can be realized with a handle design and actuator assembly that anatomically mimics human phasic, tactile mechanoreceptors that are found in skin tissue. An illustration 170 showing Meissner's corpuscles is shown in FIG. 7. In humans, Meissner's corpuscles are encapsulated unmyelinated nerve endings, which consist of flattened supportive cells arranged as horizontal lamellae surrounded by a connective tissue capsule. The corpuscle is between 30-140 μm in length and 40-60 μm in diameter. A single nerve fiber meanders between the lamellae and throughout the corpuscle. Such nerve fibers transmit neuronal signals with slow conduction speeds (slow fibers), while other cutaneous sensors and neuronal connections transmit with faster conduction times as they are myelinated (fast fibers).

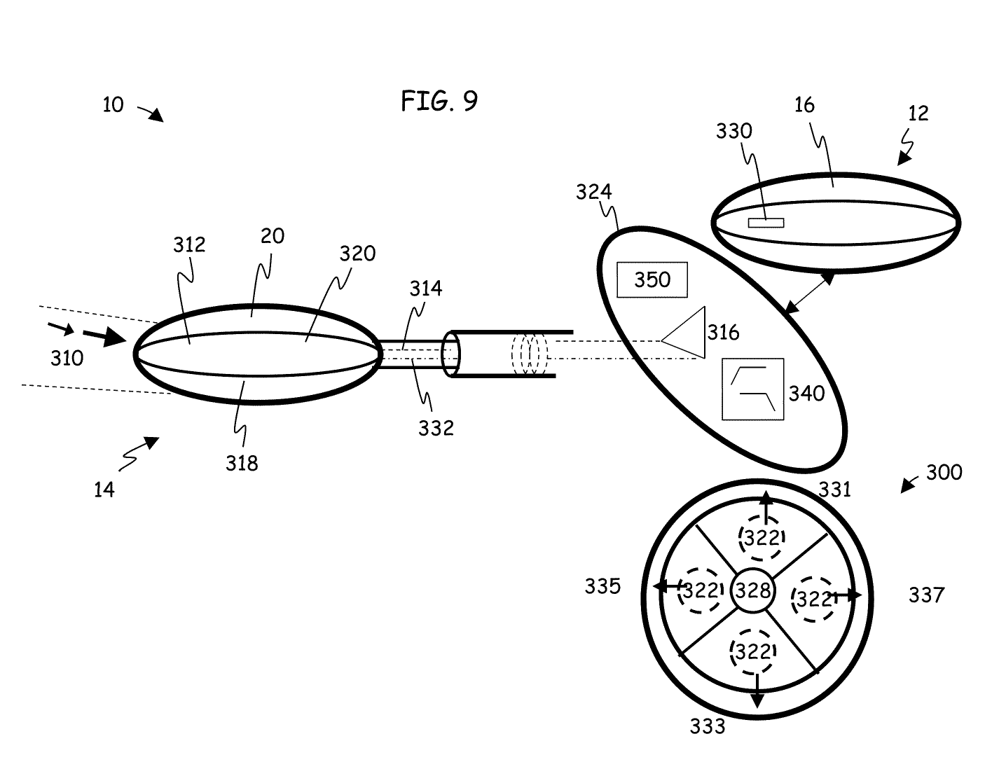

In order to enable the user to feel as if her or his hand is actual human tissue, the end effector 14 and haptic handle 12 are constructed with a design that is similar to human anatomy. FIG. 8 is illustrative of such a design and depicts fluid filled sacs 180 at the terminal ends of grouped helical nanosprings 182 with common conductors or multiple (e.g. an array) piezoactuators located at the distal end act as sensors (e.g. within or about the cryoballoon) and when displaced these nano-actuators 182 generate voltage and thus a current representative of forces and motion in situ. In this regard, one embodiment of the invention utilizes piezoelectric sensors, force and/or pressure sensors which are positioned radially about or within the inflatable ablation balloon 20 (e.g. helical nanosprings, ZincOxide(ZnO) nanowires). These acquire regional data which is conducted via conductors, nanoconductors (e.g. CNTC) or hybrid conductors (CNTC hybrid 190) which are composed of silver 192/CNTC 194 interfaces as depicted in FIG. 8. Lower profile nanoconductors will not necessitate larger diameter catheters/sheaths. The conductors transmit signals to comparable actuators (e.g., piezoactuators, smart materials, EAP) which recreate the relevant tangible sensations at the handle 12 in a regional fashion as well. Contact uniformity can be palpable at the operator's end in this fashion. A cross sectional view 200 of the handle 12 is seen in the bottom right of FIG. 8.

Global positioning systems or alternate navigational systems (e.g. electromagnetic, magnetic, admittance, thermal, impedance based, visual, handle based position sensors etc.) enable an accurate 3D representation between end-effector 14 and the haptic handle 12 during active manipulation of the handle 12 in real time. Thus, for example, superior located sensors relative to patient position are represented in the superior most quadrant 202 of the haptic handle 12. Likewise, as seen in FIG. 3, the inferior segment 204, medial segment 206 and lateral segment 208 in the haptic handle provide regional haptic displays for similarly located segments within the heart. Each regionally located sensor “innervates” regionally located actuators. The larger the number of subsegmental volumes, the more seamless the transition as the end-effector 14 and haptic handle 12 are manipulated in three dimensions.

In one embodiment, the regionally located actuators consist of ultrasonic or piezoelectric actuators with circular resonators 72 (Kern TA. Engineering Haptic Devices. (Ed.) 2009, XXXI, 472 p. 254) or linear actuators 74 that produce standing waves within each of multiple fluid filled compartments in the haptic handle 12. For example, four compartments are shown in FIG. 3. The circular resonators 72 resonate at specific frequencies dependent on the sensed events (e.g. pulmonary venous flow). Likewise, linear actuators 74 will displace a specific distance (e.g. 0-12 mm) at specific velocities dependent on the nature of sensed events (e.g. tissue displacement). The resulting standing waves provide a tangible sensation that recreates the fluid dynamic properties of sensed blood flow and cardiac motion at the haptic handle 12 qualitatively, quantitatively, spatially, and temporally. The geometric characteristics of the haptic handle 12 will reflect the anatomy of the inflated balloon 20 providing the operator with a palpable shape representative of intracardiac anatomy (e.g. proportionate cross-sectional dimensions). This will be tangible as if the operator's hand and fingers were anatomically part of the encompassing cardiac tissue and surrounding vasculature. This data can be stored for individual patients for future analysis as described below. Either the Cartesian or Polar coordinate system can be used for storing such metrics. A polar coordinate system may be more intuitive in some cases.

Parametric imaging can be used to visually display anatomic data with superimposed physiological data (e.g. elastance of pulmonary os, regional properties of the atrial antrum, pulmonary venous fluid dynamics) along with conventional imaging data (e.g. 3D CTA reconstruction).

In an alternate embodiment, the handle 12 is encompassed by a layer of conductive medium 210 such as smart fluid (e.g. magneto-rheological fluid or electro-rheological fluid) or smart material which serves as an active interface between haptic signals (e.g. standing waves) generated from juxtaposed actuators (e.g. circular resonators) coupled to the underlying handle 12, as shown in the cross sectional view 200 of the handle 12 in FIG. 8. The figure shows low frequency actuator(s) 222 surrounding high frequency actuator(s) 228. Similarly, linear actuators can be used to impart a to and fro motion comparable to displacement of cardiac tissue (e.g. atrial walls, myocardium, blood flow) which can be transmitted and conducted through a conductive medium 210. Current flow into the electro-rheological fluid 210 will cause expansion of the fluid displacing the conductive medium 210 (contained within an elastic outer shell/haptic surface 212) thereby providing the operator with force feedback representative of balloon expansion (e.g. diameter change from 10-20 mm). Subsegmental sensing and activation will enable the haptic interface to form a shape consistent with that of the end effector 14 (e.g. cryoballoon) displaying (haptic and/or visual) anatomic information. Such a haptic surface 212 can be created using alternate technologies/actuators and those discussed herein are exemplary. This type of construct will provide realistic and physiologically relevant feedback to the operator in this fashion by tempering the tactile affect from the underlying actuators with dynamic actuators sandwiched between the operator's hand and more internally located actuators.

Grant et al. describes a handheld computer interface with haptic feedback in U.S. Pat. Pub. 2011/0121953 that combines an impact actuator with smart materials to simulate various sporting activities such as hitting a tennis ball with a racquet; this publication incorporated by reference herein. The tangible sensations generated at the handle are “controlled” by the sensed internal events at the end-effector (e.g. piezoelectric sensor on distal most aspect of a guiding catheter proximate to internally deployed catheter with distally located cryoablation balloon). The application of a variety of sensors and actuators along with encompassing haptic elements about the user's hand and fingers will optimize the users experience and enable him or her to feel as if they are experiencing the forces from the inserted instrument. Thus, the sensors in their hand, fingers, wrist etc “tactually appear” to be the tissue and surrounding structures being treated and intervened on in real time. In such an embodiment, the haptic interface affects cutaneous tactile sensors, proprioceptive sensors, and kinesthetic sensors.

Any number of sensors and actuators can be used in the end effector 14 to generate the blend of tangible sensations that enable the operator to feel as if her or his fingers and hand are actually one or more segments of cardiovascular tissue (e.g. pulmonary vein endothelial lining, left atrial OS, left atrial appendage, coronary artery vessel or aortic endothelial lining etc.). Tangible sensations are sensed by activation of a multitude of cutaneous and non-cutaneous human receptors providing kinesthetic, proprioceptive and tactile sensations.

FIG. 9, illustrates a schematic depiction of the workings of an embodiment of a haptic handle system 10 including haptic handle 12 and end effector 14. In FIG. 9, pulmonary venous blood flow 310 is sensed by a piezoelectric sensor at 312, which generates voltage and thus current down conductor 314 to amplifier 316. Likewise, haptic corpuscle sensors 318 comprised of helical piezoelectric nanosprings send current down conductor 314 to amplifier 316 as does pressure or force sensor 320, which senses the force or pressure inside the inflatable balloon 20. Amplifier 316 sends the respective amplified current waveforms to reciprocating actuators in the haptic handle 12. Amplification closer to the sensors will improve signal to noise ratio, though amplification can occur anywhere in the system. Sensor combinations can be used to generate the appropriate signals to the appropriate actuators.

For example, low frequency standing wave actuators 322, depicted in the cross-sectional view 300 of the handle 12, receive signals (data) from haptic corpuscle sensors 318 and force sensors 320. These signals are band pass filtered in a control unit 324 so that a low to mid frequency range signal produces a representative haptic sensation in form of a palpable waveform that moves across the internal most surface of the handle 12, tangentially across the hand of the operator holding the handle 12. This sensation is primarily caused by one or more low frequency actuators 322 (e.g. circular resonators) that displace a liquid medium 326 (e.g. viscoelastic material, electrorheological fluid) as one or more standing wave(s) at the appropriate frequencies and with the appropriate velocity and amplitude proportionate to the sensed signal (e.g. intravascular blood flow). Accordingly, expansive movement can be made possible in the superior 331, inferior 333, medial 335, or lateral 337 directions. Higher frequency sensed signals are likewise generated by one or more high frequency actuators 328 (e.g. vibrotactile motor).

In some embodiments, inflation of balloon 20 occurs in preparation of cryoablation by an operator triggering a command switch 330. Command switch 330 can be a conventional switch, trigger and the like. In one preferred embodiment, the command switch 330 is activated by a forceful squeeze upon a portion of the handle 12 or the body of the handle 12 itself. As balloon inflation occurs at balloon 20, a reciprocating effect occurs at the expandable catheter handle haptic interface 16 of handle 12 where a current or magnetic field is generated across smart fluid or smart material 326, such as electrorheological fluid, causing a reciprocal expansion of the outer portion of the handle 12. Alternate means of providing the operator with a tangible sensation as if his or her hand were actually the endothelial surface of the contacted anatomic structure (e.g. pulmonary venous os, coronary or peripheral vasculature) are contemplated in further embodiments of the invention. In other embodiments, inflation of balloon 20 occurs automatically or semi-automatically based on time delays, sensed events or some combination thereof.

The amplitude of the degree of inflation is proportionate at the balloon 20 and the expandable haptic interface 16 of the handle 12 and correlates with the transmitting signal amplitude (e.g. current amplitude) between balloon 20 and haptic interface 16 (a synchronizing calculator serves to tune the signals as discussed in detail below). Sensing and actuation ideally occurs in multiple subsegmental volumes and provides the operator with anatomic and physiologically relevant signals reproducing the events with physiologic and anatomic accuracy occurring in situ (e.g. at the end-effector atrial interface). Force sensors 320 and/or alternate sensor technology (e.g. manometers) at the balloon 20 of end effector 14 generate one or more signals that are transmitted to the expandable haptic interface 16 of handle 12 via conductor 332 which can be amplified at amplifier 316 as needed. Thus, the operator appreciates the pulsatile properties of pulmonary venous inflow simultaneous with palpable sensations representative of balloon inflation and the properties at the interface between the balloon 20 and contacted tissue (e.g. geometry, elastance, vibration etc). Vibrotactile and displacement information due to atrial arrhythmia and atrial mechanical function is sensed at piezoelectric sensor 312 and likewise transmitted to one or more variable frequency vibrotactile actuators at 322 for low frequency data and at actuator(s) 328 for high frequency data. Band pass filtering of specific sensor signals at band pass filter 340 and processing at processor 350 serve to direct the appropriately acquired data to the appropriate actuator(s) at haptic handle 12. The higher frequency generated vibration at actuator(s) 328 matches the frequency and amplitude of sensed vibration at piezoelectric sensor 312 and/or haptic corpuscle sensor(s) 318 and is thus, reciprocating (synchronizing calculator serves to fine tune signals at the actuator).

In one embodiment, haptic rendering techniques are added to provide the operator with a tangible sensation representative of a change in temperature at balloon 20 during application of ablative energy (e.g. cryotherapy, radiofrequency energy) or to sense the high frequency chaotic effect of microbubbles during delivery of ablation energy. This is done by causing variable but high frequency vibrations via one or more of high frequency vibrotactile actuators 328 in handle 12 (psychophysical haptic rendering). Ridges on the exterior of the handle 12 will improve such haptic rendering techniques as tangential motion will be appreciated (active haptic feedback). Additional sensor technology along the surface of the handle 12 can be implemented to accomplish this. Thus, the handle 12 implements both a non-virtual haptic display with total passivity transparently recreating actual events in real time and haptic rendering techniques to provide psychophysical simulation. The former can be done purely with analog signals (as well as with digitized data) while the latter will necessitate digitization of data in processor 350 as described in more detail below.

In one embodiment, adjustment of the amplitude of the haptic effect is accomplished by modifying material properties by altering the viscosity of smart material/fluid by adjusting the current delivered to smart material/fluid. This can be done manually by the user, be programmed with user preferred default settings or in a preferred embodiment, accomplished automatically dependent on the nature of the signals driving the haptic components within the handle 12. In an advanced mode of an embodiment of the invention, mechanisms similar to those used for automatic adjustment of palpable haptic effects also serve to control or dampen application of force by the user upon biological tissues as to avoid deleterious effects (e.g., cardiac perforation). Thus, a dampening mechanism that uses smart fluid/material serves to eliminate the application of excessive force to cardiac tissue and reduces the range of force applied, eliminating peaks and valleys in force amplitude. The effects of cardiac contraction, rotation and translation as well as respiration are thus minimized in real time. Closed loop control systems, predictor algorithms and other processing techniques can be applied to adjust the viscosity of smart material/fluid within a dampening mechanism and mitigate the cyclical properties in force that would otherwise occur from patient motion, gross changes in applied force of the user upon the catheter, and the effects of the cardiac and respiratory cycles.

Processor 350 is utilized to gather acquired sensor data and process such data (e.g. A/D conversion, filtering, noise reduction). Processor 350, amplifier 316 and filter 340 can be contained in control unit 324. Control unit 324 is preferably not within the handle 12 in various embodiments, but is in a separate enclosure that is wired to, or wirelessly communicates with, handle 12. When both analog and digital signals are used for the final haptic display a time delay can be incorporated at processor 350 to adjust for the timing delays that occur due to signal processing, even though the final haptic representation will remain near instantaneous. Maintaining a natural differential in times between the onset of differing sensed signals and thus actuator generated signals will better recreate physiologically relevant tangible sensations just as slow and fast neural fibers transmit with varying conduction times. Such delays can be programmed in 350 and be based on specific characteristics of sensor generated signals (i.e. current waveforms) and dependent upon which sensors are activated (e.g. Meissner's corpuscles generated signals transmit slower than high frequency/vibrotactile signals). Both impedance and admittance type of haptic interfaces can be used (e.g. at processor 350) depending on the nature of the sensed signal and generated haptic signals such as force/motion data.

Parallel Limb Data Digitization and Storage

In order to process or store the data acquired at control unit 324 during procedures (e.g. ablation) with the technologies described herein it will be necessary to digitize the acquired analog data. In one mode of the invention, a separate, parallel circuit (parallel limb), is employed where current signals are A/D converted at 352 and processed at control unit 324. Specific metrics related to the affect of pulmonary venous flow (e.g. turbulence, laminarity, velocity, waveforms) on acquired signals at the end-effector transducers (e.g. piezoelectric sensor 312) can be derived. Likewise, corresponding tactile metrics at handle 12 (e.g. tissue displacement amplitude, blood flow velocity, acceleration, frequency and temporal characteristics, balloon inflation force) are acquired at sensors(s) 354 within or about handle 12 and then analyzed and compared to correlative signals from piezoelectric sensor 312, haptic corpuscle sensor(s) 318, force sensors 320, etc. (e.g. piezoelectric voltage, conducted current, balloon geometry during inflation, force measurements etc.).

Tactual metrics and sensed data at the end effector 14 relate directly to cardiovascular health. In particular, the acquired information can be used to analyze pulmonary venous blood flow and atrial mechanical function. Thus, a means has been designed in embodiments of the present invention for storing the data for future comparisons, especially if repeat procedures are required. Findings representative of advanced pathologic changes in pulmonary venous anatomy include higher velocity and turbulent pulmonary venous inflow (e.g. due to pulmonary venous stenosis) which would cause higher frequency, chaotic motion characteristics, as well as, pathologic changes in the geometry of the atrial antrum and pulmonary venous os, which will cause characteristic changes in the dimensions of subsegmental volumes within handle 12 and a variety of pathologic haptic representations.

By way of example, Fourier analysis, application of chaos theory and finite element analysis of acquired motion data (e.g. vibrotactile) will provide insights into specific pathophysiological processes. Data consistent with LA diastolic abnormalities will include lower amplitude displacement of antral atrial tissue during atrial systole (e.g. reflected by lower amplitude haptic signals), changes in pulmonary venous inflow patterns and changes in the elastance or compliance of a cryoballoon during inflation. Many of these pathophysiologic findings will be represented in the haptic display by changes in tangible sensations.

Storage of tangible sensation data for future analysis and comparisons will improve the diagnostic acumen of operators performing repeat procedures in patients with recurrent arrhythmia and reduce the likelihood of complications and educate clinicians as to what specific findings are indicative of specific pathologic processes (e.g. lower amplitude atrial motion, low amplitude/high frequency LAA motion indicative of higher stroke risk). Such data will also help troubleshoot diagnostic work-ups for patients who develop dyspnea or other symptoms post-procedure. Likewise the haptic representations of specific patients can be reviewed prior to repeat procedures and be made available at academic centers for educational purposes as well. Virtual recreation of pathophysiological data can be made available as a virtual reality game for data storage and education purposes and in one application, the handle 12 is implemented as a game controller, as described in more detail below.

Synchronizing Calculator/Closed Loop Control System

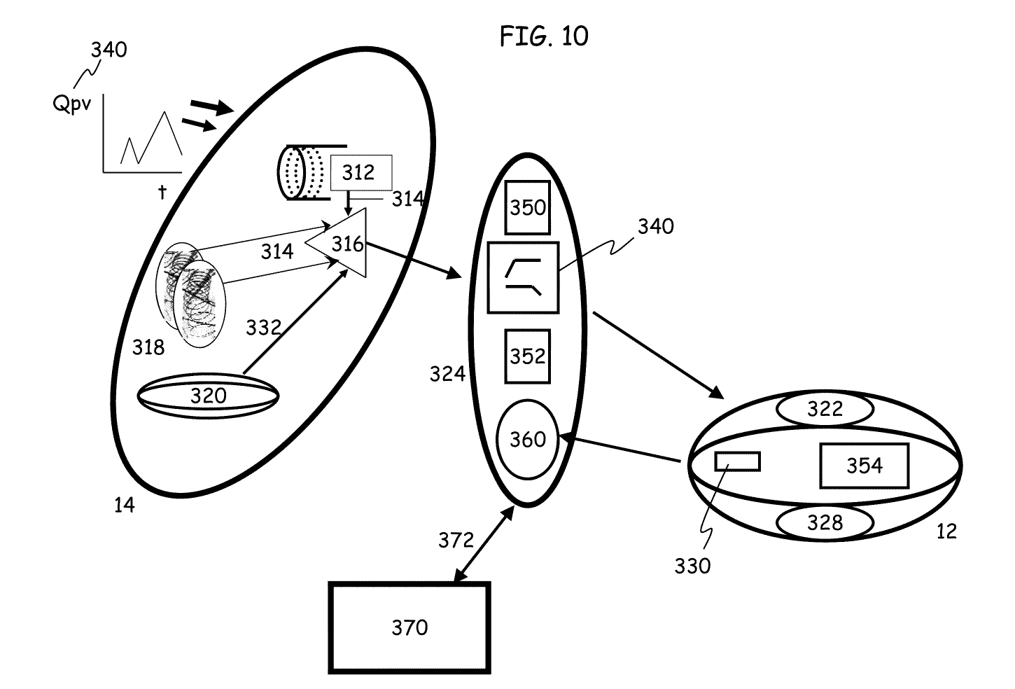

In one embodiment, a synchronizing calculator 360, is employed to perform cross comparisons between the sensed signals at end effector 14 and sensed signals at haptic handle 12, as shown in FIG. 10. The calculator 360 can be located within control unit 324. Thus, sensors within end effector 14 and the actuators within haptic handle 12 and/or additional sensors 354 (e.g., accelerometers) within haptic handle 12 are used to generate signals (e.g. current waveforms) sent to synchronizing calculator 360. At synchronizing calculator 360, data from haptic handle 12 and end effector 14 are correlated and compared. Under optimal circumstances, the respective signals are proportionate in amplitude, frequency, spectral properties etc. Fourier analysis, fractal analysis, spectral analysis and other computing techniques can be used to evaluate signals at haptic handle 12 and end effector 14 and identify salient signals for synchronization and diagnostic purposes. Processor 350 can be used to modify the behavior of actuators at haptic handle 12 and the filtering and processing at control unit 324 as to better synchronize the data between haptic handle 12 and end effector 14 using a closed loop control system. By way of example, synchronizing calculator 360 is used to ensure that the spatial and temporal quality of the tangible sensations at one or more actuators at haptic handle 12 will match that of the received signals at end effector 14 (e.g. at Meissner's corpuscles and piezoelectric material) in amplitude, velocity, force, phase etc. Specific programs and algorithms within processor 350 are used to synchronize signal sets.

The composite of data acquired at control unit 324 is used for data storage and tactile metrics are derived. Input from externally located diagnostic equipment 370 is used to better assign values of physiological significance to acquired tactile metrics derived at control unit 324 by using input auxiliary information 372 such as echo derived measurements of pulmonary venous flow, atrial mechanical function, etc. stored at diagnostic equipment 370. Alternatively, diagnostic equipment 370 can be used as the sole input of sensor data into control unit 324 effecting the handle 12, without a need for internally deployed sensor technology. In such an embodiment, diagnostic equipment 370 can be an externally located navigational system that detects the location of inserted instruments in real time (e.g. impedance based, admittance, ultrasonic, visual, electromagnetic-based etc.) or other non-contact sensor technology.

Haptic Element Uniformly Driven by Linear Actuator

Linear actuators are optimal components for driving the haptic signals in the handle 12. These can be constructed in small dimensions, have dynamic reaction times, high velocities, large stroke distances, and excellent frequency characteristics. It is recognized that individual actuators may be limited in the ability to handle side loads generated from both displacement and externally applied forces from the fingers and hand of a user (i.e. upon a mounted haptic element). In one embodiment of the invention, one or more actuators (e.g. linear actuators/stepper motors) are integrated with an apparatus designed and structured to apply a uniform load distribution to the haptic element. This can be accomplished with one or more linear actuators control unit 324. Such actuators are known by those experienced in the art and manufactured by companies such as Ultramotion LLC (Cutchogue, N.Y.).

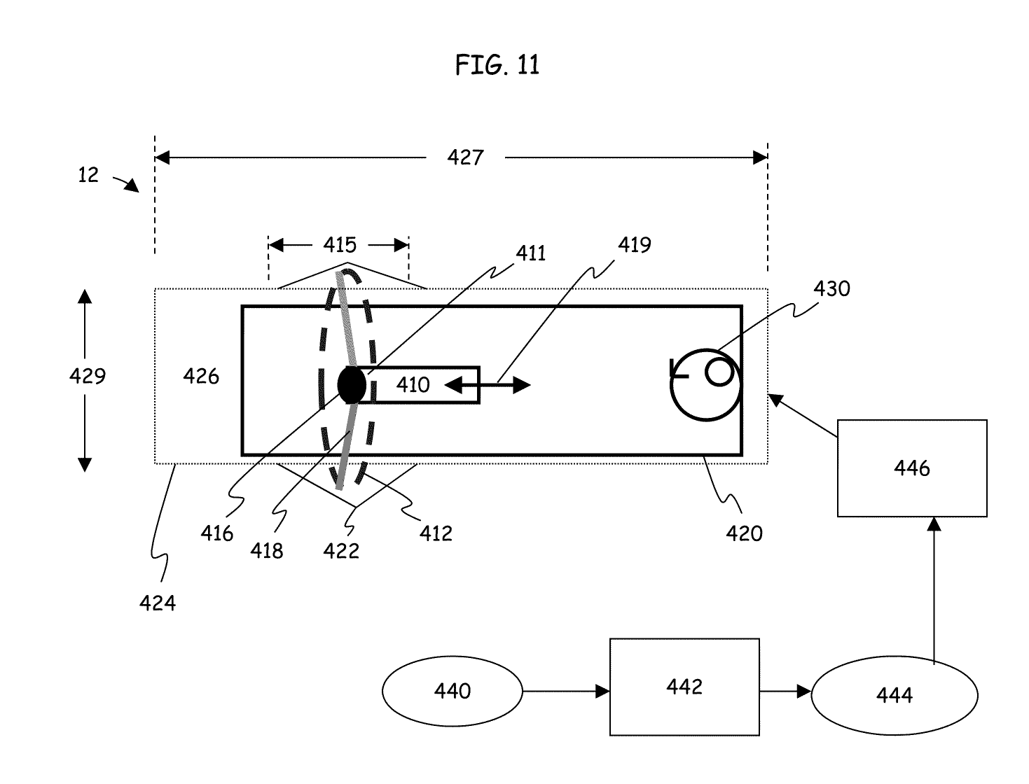

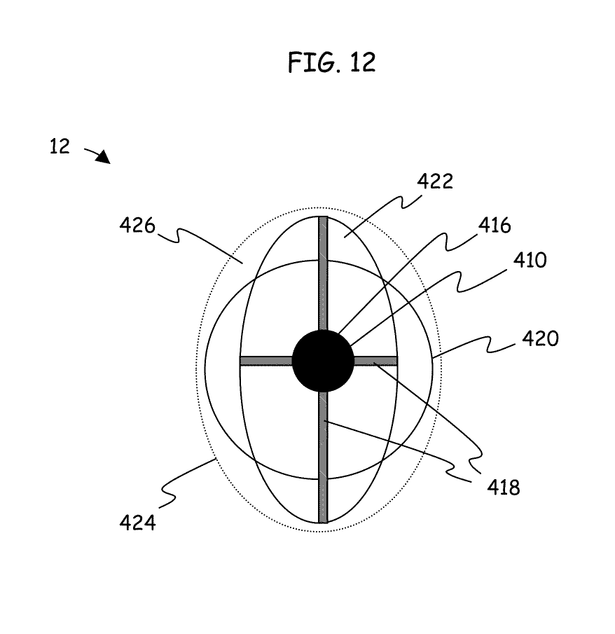

FIG. 11 shows one exemplary embodiment of a handle 12 in which a linear actuator 410 is configured to move haptic element 412 with a to and fro longitudinal motion and is driven by one or more controllers in either analog or digital format. Haptic element 412 can be a collar or knob or other structure used to provide tangible sensations to the operator. Total stroke distance 415 may be, for example, +/−10 mm (total 20 mm), velocity range may be, for example, up to 100 cm/sec, resolution may be, for example, 0.0004 inches/step. In order to provide uniform motion and load distribution to haptic element 412, the nose 411 or other portion of a single actuator 410, is mounted to a hub 416 with radially or tangentially oriented spokes 418 that support haptic element 412 in a uniform fashion. In FIG. 11, this is represented by two spokes 418 oriented outward from the central longitudinal axis 419 of linear actuator 410. Alternate support mechanisms that provide uniform distribution of imparted motion from the linear actuator 410 to haptic element 412 are within the scope and spirit of the invention. Linear actuator 410 can be contained within an inner case or housing 420 while haptic element 412 extends or is a protuberance 422 beyond this housing 420 and is encased within a compliant housing 424 filled with liquid or gel 426 that transmits the motion of the haptic element 412 to the user or via frictional effects upon a flexible haptic membrane (e.g., elastomer on surface of handle). In some embodiments, the compliant housing 424 may have a length 427 of 10-15 cm and a width 429 of 2.5-5 cm. A high frequency vibrotactile motor 430 is located within one end of the handle housing 420 in FIG. 11. The system accordingly takes sensor signals 440 and sends them to a first controller 442 for processing. Controller 442 generates a drive signal 444 that is then sent to a second controller 446 for additional processing before being relayed to the motors/actuators of the handle 12. FIG. 12 shows a cross-sectional view of the handle 12 through the knob protuberance 422.

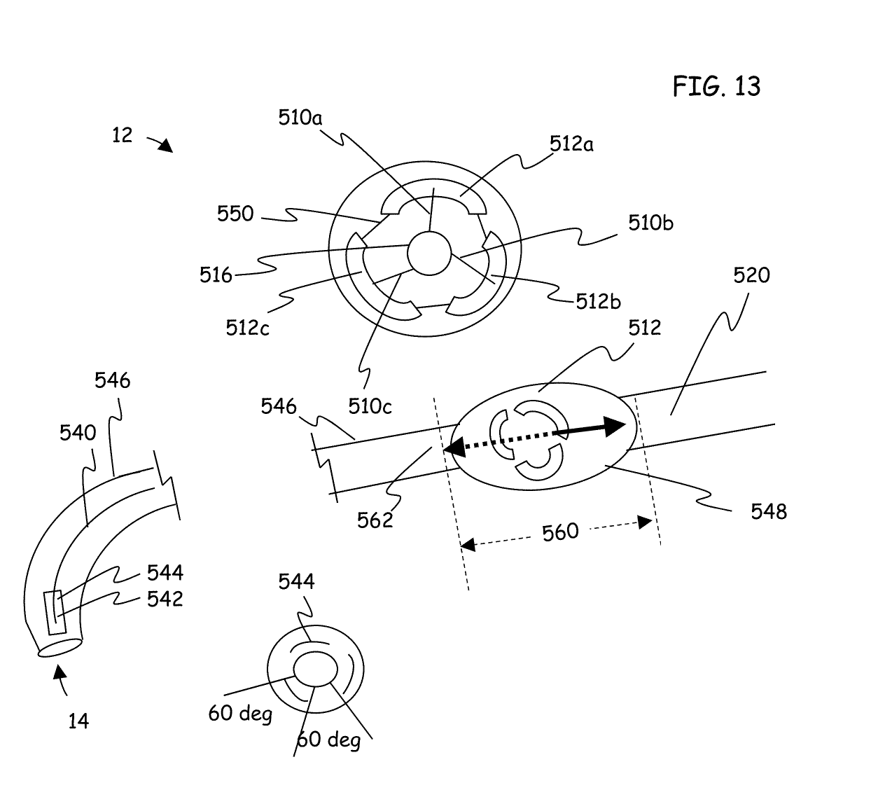

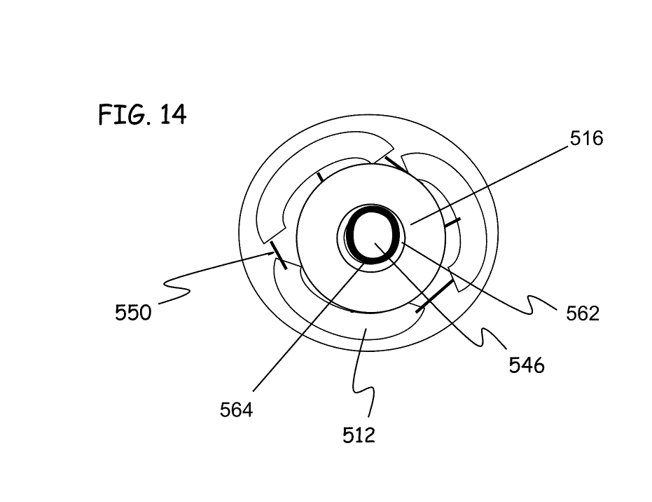

FIG. 13 sets forth an alternate embodiment, in which multiple linear actuators 510 are implemented and connected to multiple haptic elements 512 at the periphery of the handle 12 (along the haptic interface) and are joined at a central pivot or hub 516. The three linear actuators 510a, 510b, and 510c (or collectively 510) have center points oriented 120 degrees apart which support and drive three circumferentially positioned haptic pads 512a, 512b, and 512c (or collectively 512) about a stator shaft/handle 520. In one exemplary embodiment, each of three linear actuators 510 (tri-axial configuration) are connected with separate conductors 540 to tri-axial haptic pads 542a, 542b, and 542c (or collectively 542) driven or innervated by a different sensor 544 which is also positioned in a tri-axial fashion on the end-effector 14 (e.g. as depicted sealed sensors 544 layered on a conductive silver surface embedded upon the distal aspect of catheter 546). Thus, the haptic element(s) 512 provide the user with a volumetric haptic display. Piezoelectric or other (e.g. strain gauge, peizotronic, piezoactuators, etc.) sensors 544, in one embodiment, are embedded upon or within the catheter 546, each spanning 60 degrees and radially separated from neighboring sensors 544 by 60 degrees or other spacing dependent on the sensors surface areas, such that the center point for each sensor is 180 degrees apart. These sensors need not be located in the same axial location along the catheter and can be separated by a specified distance (e.g. 5-50 mm) so that they are situated along a spiral coursing along the longitudinal axis of the catheter. Other geometric configurations, and variable numbers of sensors and actuators are within the scope and spirit of embodiments of the invention. By way of example, each haptic pad 512a, 512b, and 512c (or 542a, 542b, and 542c) can be separated or connected to one another with a circular connecting rod 550. The rod 550 can be fabricated with shape memory alloy or other material that is deformable and becomes more compliant when held by the operator and warmed to body temperature or be rigid and non-deformable. The deformable construct will provide a more accurate recreation of the in situ environment as the distal aspect of the catheter 546 becomes more compliant during insertion into a patient and the properties of a deformable material (e.g., shape memory alloy). This will serve to provide the operator with a real feel simulation of contacted tissue opposed to an inserted catheter that has similar temperature dependent changes in its deformation properties. The space 548 between all the actuators and haptic elements can be empty or filled with gas, liquid or other material that protects the components or acts as an active component (e.g. smart fluid/material) that provides haptic feedback. The components (e.g., 512, 542, 550, etc.) can function individually, or in one embodiment interact with one another, for example, mechanically (e.g., via rod 550), electrically (conductive rod 550), magnetically (e.g., via EMF generated by linear actuators 510), pneumatically or hydraulically. All the components are encased within housing of the handle 12 and, in total, are sized to fit comfortably within the palmar grasp of the human hand. Varying dimensions of the housing will suit individual user's preferences. In some embodiments, the length 560 of the haptic portion of the housing is generally between 80-180 mm. In one embodiment, a haptic catheter 562, used to guide the inserted catheter 546 through the handle, courses through a centrally located port or conduit within the central hub 516. This arrangement can be seen in the cross-sectional view of FIG. 14. The conduit is juxtaposed to, and centrally positioned at a center point between all the inner components. This design will ensure that haptic effects are imparted to the haptic catheter 562 and inserted catheter 546. A deformable sealing ring 564, or alternate construct, allows for variably sized haptic catheters 562 and guiding catheters 546 to fit snugly and function within the haptic handle 12. Alternate constructs for positioning the haptic catheter 562 through the haptic section of the handle and imparting haptic effects to the catheters used to perform procedures are within the scope and spirit of embodiments of the invention, including but not limited to a construct where haptic catheter 562 is off the central axis, peripherally located, or where one or more portions of the handle 12 are attachable/detachable, or even located in another location and controlled telerobotically.

Quad Mode Haptic Actuators

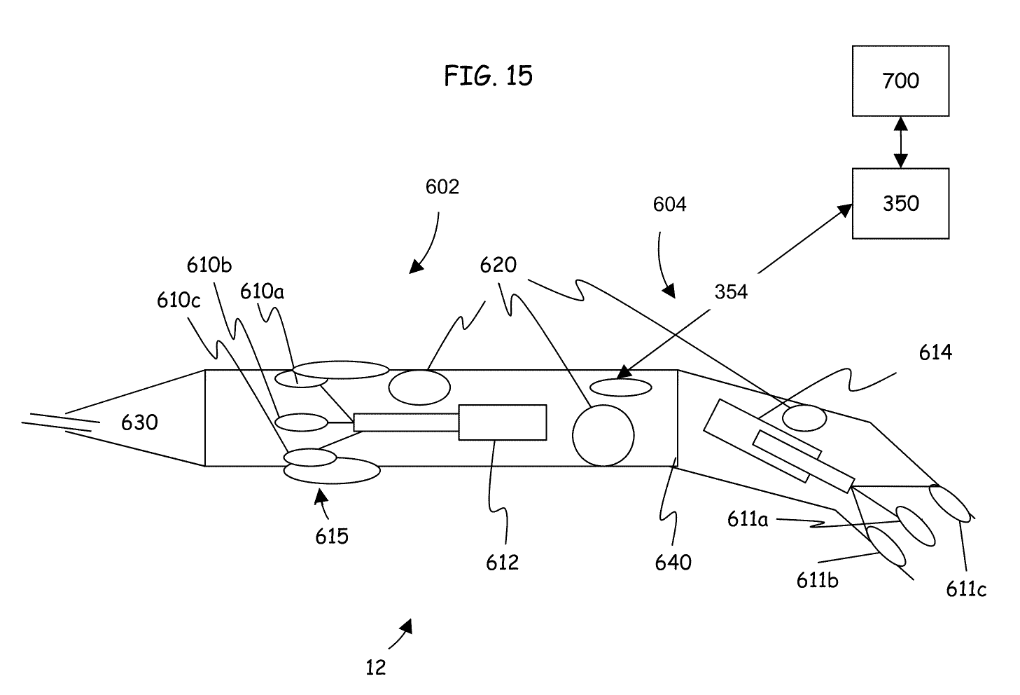

In still yet another embodiment, the haptic element(s) is/are operational in one of four modes. A static mode where there is no actuation or haptic feedback and haptic element is neither haptic nor actuating, a haptic mode where haptic element provides haptic feedback, an actuating mode where haptic element is used to deform or maneuver one or more aspects of the catheter as known by those experienced in the art and a haptic-actuating mode where haptic element provides haptic feedback and is used to deform and modify the characteristics of the inserted catheter. The user can switch modes depending on individual preferences. The gain of haptic feedback is adjustable and when programmed to zero gain haptic element will not provide haptic feedback. Various handles can be used during interventional procedures and exchanged as needed. One embodiment of the invention includes a lower cost handle that does not provide haptic feedback but is equipped with a means for acquiring regional sensor data from the inserted end-effector of the balloon and optimizing regional inflation pressure at 20 as depicted in FIG. 4 and described above. This handle can be exchanged or be used in place of the haptic interface and attached to the inserted catheter, for example, at 162 as depicted in FIG. 5 (or even controlled telerobotically). Such an embodiment will allow for delivery of therapy once an optimal anatomic position is confirmed and is essentially the same as having zero haptic gain with a lower cost handle but enables manual, semi-automatic and automatic control over regional balloon insufflation. Closed loop control over regional balloon insufflation pressure occurs in processor 350, as depicted in FIG. 15.

Electrical and Contact Uniformity Confirmation System

In another embodiment of the invention, sensed intra-cardiac electrical depolarizations at the end-effector 14 are palpable at the haptic handle 12. By way of example, electrodes positioned at the distal end of the inserted catheter/instrument 546 detect localized electrical activity. Such sensed intra-cardiac electrograms confirm tissue contact, provide diagnostic information and identify when specific pathways are interrupted (e.g. after ablation about the pulmonary veins).

Referring to FIG. 1, sensor strips 22 in this embodiment are representative of multiple electrodes. Such electrodes can be used for both sensing mechanical properties, and electrical depolarizations. Sensed data (electrical and mechanical) are represented at the handle 12 and signify uniform tissue contact, electrical activation patterns and tissue characteristics. Preferably, the handle 12 is similarly shaped (e.g. as a deformable balloon shape) and tangible sensations are indicative of both tissue physical properties (e.g. motion) and electrical activation. The former can be manifest as motion in the handle 12 and the latter with tangible sensations simulating electrical depolarization using haptic rendering techniques (e.g. regional changes in low amplitude high frequency vibration). Such an embodiment will assist the operator in identifying the location of anatomic structures (e.g. LAA, venous os, cardiac valves) by palpation by detection of gradients in amplitude of frequency, vibration, geometry, displacement, tissue and blood flow velocity, laminarity, turbulence and acceleration, as well as, electrical characteristics. When a rigid instrument is used (as opposed to a deformable catheter), flexible material interspersed or in parallel to the more rigid material (e.g. metal alloys used in robotic systems) or semi-rigid materials can be used to better acquire low frequency motion/vibration data representative of tissue motion and blood flow (e.g., elongated piezoactuator tubes).

Bimanual Handle Design

In an embodiment shown in FIG. 15, the handle 12 is composed of two sections 602 and 604, which enable the user to utilize both hands for palpation of physiologically relevant tangible sensations. The housing containing all the components of handle 12 is preferably spherical or curved in geometry. Variable frequency vibrotactile motors, linear actuators and other mechanisms for generating relevant physiologic signals are present in both sections, each section displaying tactual data from distal and proximal sections of an inserted catheter/instrument, that improve the operator's ability to palpate the spatial and temporal recreation of tissue properties, and the affect of the inserted catheter on tissue properties and intracardiac/intravascular blood flow. A textured haptic sleeve (not depicted) may be used to enhance perception of subtle haptic signals (e.g., shear force, friction effects, tangential motion) and contain ribs, bumps and the like. Such a disposable sleeve 650 can be composed of any type of elastic material that will protect the handle 12 from the environment and enhance tangible sensations.

Referring to FIG. 15, an embodiment of one example of construction for the bimanual handle with haptic feedback is shown. Linear actuator 612 is connected to haptic elements 610a, 610b and 610c that provide tangible sensations to an operator as they course to and fro along the undersurface of haptic membrane 615. The haptic membrane 615 is an elastic, thin polymer that is situated in a location that will be opposed to the palmer surface of the hand of a user most proximate to the patient (usually the left hand). Likewise, a similar configuration implementing haptic elements 611a, 611b, and 611c and linear motor 614, is located for the hand of a user most distal to the patient (usually the right hand). Additional actuators, such as rotary motors with offset weights 620, are located within the handle, 12. The section 630, nearest the patient and entry point of inserted/catheter instrumentation, assists in directing the catheter assembly into the body of the patient. If section 630 is not needed, it is able to be detached, for example, if handle 12 is used telerobotically. This and other sections of the handle 12 can be detachable via mechanisms as understood by those experienced in the art at specific locations, using connectors 640, if specific users desire to only use a one-handed handle configuration. Sections (within or about the outer surface) of the handle 12 can contain sensors such as accelerometers 354, which acquire data about the motion of the handle and communicate this data to processor 350 as part of the closed loop control system. The data is displayed at user interface 700 for the operator to review and in a preferable embodiment, enables programming of haptic effects and user control over system function as needed. As depicted, the bimanual handle is preferably not linear but is curved like a steering wheel with a radius that can be fixed at connector 640 or adjustable to suit the preferences of an individual user. In one configuration, the proximate section 602 of the handle 12 is spherical similar to the shape of a balloon and haptic membrane 615 is located along the perimeter of this section, while the distal section 604 of the handle is more tubular in construct and reflects the structure of the catheter portion of the catheter assembly.

This volumetric, bimanual haptic handle display will enable multiple sensors and sensor types (e.g., pressure, force, strain, displacement, etc.) on the inserted catheter assembly and attached end effector to correspond to multiple actuators on the handle thereby providing the user with a realistic recreation of events in vivo. Such an advanced design will enable an operator to appreciate the complex interaction between inserted instrumentation and moving biological tissue in real time including the effects of contacting and crossing tissue planes (e.g., interatrial septum), and modifications that are made to anatomic structures, physical and physiological properties of tissue, and blood flow properties (e.g., stent deployment, left atrial appendage occlusion, valvular replacement/repair) as therapeutic interventions are delivered.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

It should also be appreciated that the exemplary embodiment or exemplary embodiments are only examples, and are not intended to limit the scope, applicability, or configuration of the invention in any way. Rather, the foregoing detailed description will provide those skilled in the art with an enabling disclosure for implementing the exemplary embodiment or exemplary embodiments. It should be understood that various changes can be made in the function and arrangement of elements without departing from the scope of the invention as set forth in the appended claims and the legal equivalents thereof.

The embodiments above are intended to be illustrative and not limiting. Additional embodiments are within the claims. Although the present invention has been described with reference to particular embodiments, workers skilled in the art will recognize that changes may be made in form and detail without departing from the spirit and scope of the invention.

Various modifications to the invention may be apparent to one of skill in the art upon reading this disclosure. For example, persons of ordinary skill in the relevant art will recognize that the various features described for the different embodiments of the invention can be suitably combined, un-combined, and re-combined with other features, alone, or in different combinations, within the spirit of the invention. Likewise, the various features described above should all be regarded as example embodiments, rather than limitations to the scope or spirit of the invention. Therefore, the above is not contemplated to limit the scope of the present invention.

For purposes of interpreting the claims for the present invention, it is expressly intended that the provisions of Section 112, sixth paragraph of 35 U.S.C. are not to be invoked unless the specific terms “means for” or “step for” are recited in a claim.

1. A haptic handle system for balloon-tipped catheter interventions that provides real-time haptic feedback in the form of tangible sensations to an operator, comprising:
a catheter assembly including:
a catheter including an elongate body having a proximal end and a distal tip, and
an end effector, including an interventional balloon having a plurality of sensors, proximate the distal tip of the catheter;
a haptic handle coupled to the proximal end of the catheter, the haptic handle including a haptic interface that exhibits geometric characteristics reflecting an anatomy of the interventional balloon and provides tangible sensations representative of tissues surrounding the interventional balloon; and
a control unit operably connected to the catheter assembly that acquires data from the plurality of sensors and directs actuation of the haptic interface.

2. The haptic handle system of claim 1, wherein the distal tip of the catheter is configured to contact human tissue and deliver therapy as one or more of: hot or cold thermal energy, radiofrequency energy, electromagnetic energy, and expansive forces.

3. The haptic handle system of claim 1, wherein the distal tip of the catheter is fitted with a plurality of inflatable balloons for delivery of therapy to tissue.

4. The haptic handle system of claim 1, wherein the interventional balloon is fitted with sensors that acquire information about the uniformity of contact with the tissue being treated and forces at the interface between the balloon and contacted tissue and represent this in the handle using haptic feedback.

5. The haptic handle system of claim 1, wherein the handle is configured to permit the operator holding the handle to palpably appreciate one or more forces due to balloon inflation, contact uniformity of balloon inflation with treated tissue and degree of balloon inflation and in response manually, automatically and/or tele-robotically cause regional adjustment of delivered therapy.

6. The haptic handle system of claim 1, wherein said handle is equipped with a mechanism configured for the operator to control balloon inflation with one or more controllers located on the handle with one or more of; electrical, pneumatic or hydraulic methodologies.

7. The haptic handle system of claim 1, wherein the sensors are composed of sensor material disposed as part of an inflatable balloon to sense properties of tissue proximate the distal tip of the catheter.

8. The haptic handle system of claim 7, wherein the sensors are one or more of: circumferentially or longitudinally positioned sensors about the balloon; sensors disposed of in between layers of material that constitute the balloon; sensors used to fabricate one or more portions of the balloon, and sensors tolerant of thermal, radiofrequency or electromagnetic energy used to ablate tissue.

9. The haptic handle system of claim 7, wherein the sensors are used to measure physical and mechanical characteristics that include one or more of force, pressure, strain, displacement, velocity, acceleration, blood flow and electrical activation.

10. The haptic handle system of claim 1, wherein forces and vibrations of the haptic handle system are reciprocal between the interventional balloon of the catheter assembly and the balloon-shaped haptic interface of the haptic handle.

11. A haptic handle for balloon-tipped catheter interventions that provides real-time haptic feedback in the form of tangible sensations to an operator holding the handle, comprising:
an expandable balloon-shaped haptic interface that exhibits geometric characteristics reflecting an anatomy of a corresponding inflated balloon of a balloon-tipped catheter and sensations representative of the properties of tissue and bodily fluids surrounding the corresponding inflated balloon, wherein the expandable balloon-shaped haptic interface includes a plurality of actuators.

12. The haptic handle of claim 11, wherein the expandable balloon-shaped haptic interface provides haptic feedback of therapy deliveries of hot or cold thermal energy, radiofrequency energy, electromagnetic energy, expansive forces, or forces used to treat diseased cardiac or vascular tissues.

13. The haptic handle of claim 11, wherein the expandable balloon-shaped haptic interface indicates haptic feedback information about the uniformity of tissue contact and forces at the interface between the inflated balloon and contacted tissue.

14. The haptic handle of claim 11, wherein the expandable balloon-shaped haptic interface provides palpable sensations of one or more forces due to balloon inflation, contact uniformity of balloon inflation with treated tissue, and degree of balloon inflation.

15. The haptic handle of claim 14, wherein the haptic handle is configured to cause regional adjustment of delivered therapy selectively responsive to manual, automatic and/or tele-robotic inputs.

16. The haptic handle of claim 11, wherein the haptic handle includes a mechanism configured for an operator to control balloon inflation with a controller located on the haptic handle with one or more of electrical, pneumatic and hydraulic methodologies.

17. A haptic handle system for balloon-tipped catheter interventions that provides real-time haptic feedback in the form of tangible sensations to an operator, comprising:
a catheter assembly including:
a catheter including an elongate body having a proximal end and a distal tip, and
an interventional balloon located proximate the distal tip of the catheter; and
a bimanual haptic handle coupled to the proximal end of the catheter, the haptic handle including a haptic interface that exhibits geometric characteristics reflecting an anatomy of the interventional balloon and provides tangible sensations representative of tissues surrounding the interventional balloon, including means to impart haptic feedback to both hands of the operator wherein the haptic feedback is imparted to a first more distally located section for one hand and a second more proximally located section for another hand.

18. The haptic handle system of claim 17, wherein the first more distally located section of the handle is detachable and adapted for variable orientation relative to the second more proximally located section of the handle.

19. The haptic handle system of claim 17, wherein the haptic feedback is representative of the geometry or mechanics of one or more of the coronary sinus, pulmonary veins, intracardiac chambers, vascular and cardiovascular tissue.

20. A method of operation for a haptic handle system, comprising:
providing a haptic handle for balloon-tipped catheter interventions, including an expandable balloon-shaped haptic interface with a plurality of actuators;
reproducing geometric characteristics reflecting an anatomy of a corresponding inflated balloon of a balloon-tipped catheter and sensations representative of the properties of tissue surrounding the corresponding inflated balloon with the expandable balloon-shaped haptic interface in real-time.
